SEQUENCE LISTING (28 sequences)

SEQ ID NO: 1 - DNA, 18 bp, Artificial Sequence
  Synthetic
gcctgcctctccgctcac

SEQ ID NO: 2 - DNA, 23 bp, Artificial Sequence
  Synthetic
atctcttcgtcttcctcgtcctc

SEQ ID NO: 3 - DNA, 20 bp, Artificial Sequence
  Synthetic
gctgaatctccaagcctcgt

SEQ ID NO: 4 - DNA, 20 bp, Artificial Sequence
  Synthetic
tgttttcgcccaacatgcag

SEQ ID NO: 5 - DNA, 20 bp, Artificial Sequence
  Synthetic
acggtctgctctgggtttcc

SEQ ID NO: 6 - DNA, 23 bp, Artificial Sequence
  Synthetic
atcttggacttgagtgctgttgg

SEQ ID NO: 7 - DNA, 20 bp, Artificial Sequence
  Synthetic
ggcgtcaacaacctcactgg

SEQ ID NO: 8 - DNA, 24 bp, Artificial Sequence
  Synthetic
tgaacctgtcctttgtgtatctgc

SEQ ID NO: 9 - DNA, 20 bp, Artificial Sequence
  Synthetic
tgcccgctccaaggtgtatc

SEQ ID NO: 10 - DNA, 19 bp, Artificial Sequence
  Synthetic
gagaccgtcctcccgaagc

SEQ ID NO: 11 - DNA, 18 bp, Artificial Sequence
  Synthetic
gcctgcctctccgctcac

SEQ ID NO: 12 - DNA, 23 bp, Artificial Sequence
  Synthetic
atctcttcgtcttcctcgtcctc

SEQ ID NO: 13 - DNA, 21 bp, Artificial Sequence
  Synthetic
ccaaatgccagggaacgactc

SEQ ID NO: 14 - DNA, 20 bp, Artificial Sequence
  Synthetic
gtctgaggagccgaggaagc

SEQ ID NO: 15 - DNA, 22 bp, Artificial Sequence
  Synthetic
agagtgggctgaagtgaaggag

SEQ ID NO: 16 - DNA, 19 bp, Artificial Sequence
  Synthetic
cggttgcgggagatgatgc

SEQ ID NO: 17 - DNA, 20 bp, Artificial Sequence
  Synthetic
gcgcctttctatctgtccgt

SEQ ID NO: 18 - DNA, 20 bp, Artificial Sequence
  Synthetic
cacagtcttcctcgtcggtg

SEQ ID NO: 19 - DNA, 20 bp, Artificial Sequence
  Synthetic
ggcaggtgtagcctttctga

SEQ ID NO: 20 - DNA, 20 bp, Artificial Sequence
  Synthetic
tcagtccgctctgcaaactc

SEQ ID NO: 21 - DNA, 20 bp, Artificial Sequence
  Synthetic
acggtctgctctgggtttcc

SEQ ID NO: 22 - DNA, 23 bp, Artificial Sequence
  Synthetic
atcttggacttgagtgctgttgg

SEQ ID NO: 23 - DNA, 20 bp, Artificial Sequence
  Synthetic
atctgcatccaccaaccctg

SEQ ID NO: 24 - DNA, 20 bp, Artificial Sequence
  Synthetic
gctgaatctccaagcctcgt

SEQ ID NO: 25 - DNA, 21 bp, Artificial Sequence
  Synthetic
agcctcaacgactccatccag

SEQ ID NO: 26 - DNA, 19 bp, Artificial Sequence
  Synthetic
gtgacagcgggaaccttgg

SEQ ID NO: 27 - DNA, 20 bp, Artificial Sequence
  Synthetic
cacacaacgcccatcctctg

SEQ ID NO: 28 - DNA, 22 bp, Artificial Sequence
  Synthetic
cctgaatgcctctgaagacacc

(19) United States
(12) Patent Application Publication
(10) Pub. No.: US 2023/0340406 A1
(43) Pub. Date: Oct. 26, 2023

(54) METHODS AND COMPOSITIONS FOR PRODUCING STEM CELL DERIVED SPINAL GABA INHIBITORY NEURONS FOR USE IN TREATMENT OF SPINAL CORD INJURY

(71) Applicant: BrainXell, Inc., Madison, WI (US)

(72) Inventor: Zhong-Wei Du, Middleton, WI (US)

(21) Appl. No.: 18/013,642

(22) PCT Filed: Jul. 1, 2021
(86) PCT No.: PCT/US2021/040178

§ 371 Date: Dec. 29, 2022

Related U.S. Application Data
(60) Provisional application No. 63/047,697, filed on Jul. 2, 2020.

(51) Int. Cl. C12N 5/0793; A61K 35/30; A61P 25/00

(52) U.S. Cl. CPC C12N 5/0619; A61K 35/30; A61P 25/00

(57) ABSTRACT

Disclosed herein are in vitro methods of inducing differentiation of human stem cells into spinal GABA interneurons, and spinal dorsal interneurons domain 4 (dl4) progenitors thereof, and cells generated by such methods. The presently disclosed subject matter also provides for uses of such cells for treating spinal cord injury.

### CROSS-REFERENCE TO RELATED APPLICATIONS

This application claims priority to U.S. Provisional Patent Application No. 63/047,697, filed Jul. 2, 2020, which is incorporated herein by reference in its entirety.

### FIELD

This disclosure relates to methods and compositions for producing a population of spinal GABA interneurons differentiated from human pluripotent stem cells, in particular, spinal dorsal interneurons domain 4 (dl4) GABA interneurons, and uses thereof for cell-based treatment of spinal cord injury.

### INTRODUCTION

Spinal cord injury (SCI) is one of the leading causes of disability. It results in chronic motor deficit or even paralysis and is often accompanied by complications such as muscle spasticity and pain, which in turn perpetuate locomotion deficits. Substantial research efforts have been made to develop novel therapies for SCI. Cell transplantation has emerged as a potential strategy to repair the injured spinal cord. Numerous cell types have been assessed for their capacity to treat SCI, including Schwann cells, neural stem and progenitor cells, oligodendrocyte precursor cells (OPCs), olfactory ensheathing cells (OECs), and mesenchymal stem cells (MSCs). These cells are thought to bridge the injured cord, and provide neuroprotection and myelination. The main objective of cell therapy is to restore locomotion, and in some cases, mitigate pain. Less attention is paid to reducing muscle spasticity and neuropathic pain, the chief complications of SCI.

Spasticity, or involuntary muscle contraction, presents in 94% of the cervical and thoracic SCI patients and in 5% of lumbar/sacral SCI patients The cause of spasticity after SCI includes the loss of the inhibitory somatosensory interneurons, down-regulation of KCC2, and increased expression of 5-HT2c receptors in spinal dl4s, all of which result in over excitation of spinal dl4s. Hence, reversal of the inhibition to excitation imbalance on spinal dl4s, such as by transplanting the GABA inhibitory neurons, may mitigate spasticity. There is a need for methods to obtain the necessary cells for therapy and for improved treatments for SCI.

### SUMMARY

In an aspect, the disclosure relates to a method of generating a population of spinal dl4 progenitor cells from human stem cells. The method may include (a) culturing the human stem cells in a culture medium comprising a Wnt signaling pathway agonist, a BMP signaling pathway inhibitor, and a TGFβ signaling pathway inhibitor, to generate a population of cells comprising spinal neuroepithelia progenitors, wherein at least 95% of the population of cells generated are Sox1+/Hoxa3+ spinal neuroepithelia progenitors; and (b) culturing the spinal neuroepithelia progenitors of step (a) in a medium additionally comprising a retinoic acid (RA) signaling agonist and a SHH signaling inhibitor, to generate a population of cells comprising spinal dl4 progenitor cells, wherein at least 90% of the population of cells generated are PTF1A+/PAX7+/ASCL1+ spinal dl4 progenitor cells.

In some embodiments, the human stem cells are human pluripotent stem cells. In some embodiments, the human pluripotent stem cells are human embryonic stem cells or human induced pluripotent stem cells. In some embodiments, the human stem cells are obtained from a human embryo. In some embodiments, the Wnt signaling pathway agonist comprises a GSK3 inhibitor selected from the group consisting of CHIR99021 and 6-bromo-iridium-3′-oxime. In some embodiments, the BMP signaling pathway inhibitor is selected from the group consisting of DMH-1, Dorsomorphin, and LDN-193189. In some embodiments, the TGFβ signaling pathway inhibitor is selected from the group consisting of SB431542, SB505124, and A83-01. In some embodiments, the RA signaling agonist is selected from the group consisting of retinoic acid (RA) and EC23. In some embodiments, the SHH signaling inhibitor is selected from the group consisting of cyclopamine and SANT-1. In some embodiments, the culture medium of step (a) comprises 2 μM SB431542, 2 μM DMH1, and 3 μM CHIR99021. In some embodiments, the human stem cells are cultured in the culture medium in step (a) for 5 to 10 days. In some embodiments, the human stem cells are cultured in the culture medium in step (a) for 7 days. In some embodiments, the culture medium of step (b) comprises DMH-1, SB431542, CHIR99021, all-trans retinoic acid, and cyclopamine. In some embodiments, the culture medium of step (b) comprises about 1 μM-10 μM DMH-1, about 1 μM-10 μM SB431542, about 1 μM-10 μM CHIR99021, about 0.01 μM-2 μM RA, and about 0.1 μM-2 μM cyclopamine. In some embodiments, the culture medium of step (b) comprises 2 μM SB431542, 2 μM DMH1, 3 μM CHIR99021, 0.1 μM all-trans retinoic acid, and 0.5 μM cyclopamine. In some embodiments, the spinal neuroepithelia progenitors are cultured in the culture medium in step (b) for 5 to 10 days. In some embodiments, the spinal neuroepithelia progenitors are cultured in the culture medium in step (b) for 7 days. In some embodiments, the spinal dl4 progenitor cells of step (b) express at least one gene selected from PTF1A, PAX7, and ASCL1, or a combination thereof. In some embodiments, at least a portion of the spinal dl4 progenitor cells of step (b) further differentiate into spinal GABA interneurons. In some embodiments, the spinal GABA interneurons express at least one gene selected from PAX2 and LHX1/5 or a combination thereof.

In a further aspect, the disclosure relates to a method for treating a neurological disorder in a subject. The method may include administering to the subject an effective amount of a population of spinal dl4 progenitor cells generated by the method of generating a population of spinal dl4 progenitor cells from human stem cells as detailed herein. In some embodiments, the neurological disorder comprises a spinal cord injury.

The disclosure provides for other aspects and embodiments that will be apparent in light of the following detailed description and accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow chart depicting directed differentiation of spinal dl4 progenitors and GABA interneurons from pluripotent stem cells. Abbreviations: PSC (pluripotent stem cell); dl4 (dorsal interneurons domain 4).

FIG. 2A, FIG. 2B, FIG. 2C, FIG. 2D, FIG. 2E, FIG. 2F, FIG. 2G, FIG. 2H, and FIG. 2I show differentiation of dl4 spinal GABA neurons from hPSCs. FIG. 2A is a schematic diagram showing the transcriptional code for dorsal spinal progenitors and post-mitotic neurons. FIG. 2B shows a scheme for differentiation of hPSCs to spinal GABA neurons. SB=SB431542, CHIR=CHIR99021, RA=retinoic acid, CYC=cyclopamine. FIG. 2C-FIG. 2G show immunostaining of differentiated neural progenitors at D14 (FIG. 2C-FIG. 2E) and post-mitotic neurons at D21 (FIG. 2FC-FIG. 2G). FIG. 2H shows results from RT-qPCR showing expression of dl4 -related transcription factor genes NGN1, NGN2, LBX-1, and PAX2; dl1-3 associated genes BRN3A, MATH1, TLX3, and ISL-1; dl5-6 related transcription factor genes LMX1B, WT1, TLX3, and BHLHB5; and v1 regional gene EN1. FIG. 2I shows quantification of cell populations that express HOXA3, PTF1A, PAX7, ASCL1, PAX2/LHX1/5, and GABA in cells differentiated from hESC (H9) and iPSC (IMR-90). Scale bar: 50 μm.

FIG. 3A, FIG. 3B, FIG. 3C, FIG. 3D, FIG. 3E, and FIG. 3F show patch-clamping electrophysiological recording on differentiated cells. FIG. 3A shows the outward K+ and inward Na+ currents evoked by electrical stimulation from −70 mV to +100 mV in 10 mV increments. FIG. 3B shows action potentials evoked by current steps from −30 pA to +50 pA in 10 pA increments. FIG. 3C-FIG. 3E are representative traces of typical firing patterns shown as tonic firing, delayed firing, and initial bursting. FIG. 3F shows statistical analysis of the action potential amplitude in different firing patterns.

FIG. 4A, FIG. 4B, FIG. 4C, FIG. 4D, FIG. 4E, FIG. 4F, FIG. 4G, FIG. 4H, FIG. 4I, and FIG. 4J show survival and differentiation of transplanted human cells. FIG. 4A shows the experimental scheme depicting the schedule for SCI, cell transplantation, and behavioral analyses. FIG. 4B shows the cavity size expressed as a percentage of the coronal slices of the spinal cord in SCI (11.14%±1.3%), SCI+Medium (10.95%±1.96%), and SCI+Cell groups (5.93%±0.79%). FIG. 4C shows the distribution of hNu and mCherry double positive cells in the spinal cord rostral to the lesion cavity. Note there is still a small cavity in the center of the section. FIG. 4D shows a stereological quantification of the total number of hNu+ cells at 6 weeks and at 12 weeks post-transplantation (n=6 at each time point). FIG. 4E shows the quantification of Ki67+ dividing cells among hNu+ grafted cells at 6 weeks and 12 weeks post-transplantation as well as in endogenous spinal cord cells of sham animals. FIG. 4F-FIG. 4H show co-immunostaining of hNu with NeuN (FIG. 4F), DCX (FIG. 4G), and GABA (FIG. 4H) in the spinal cord 12 weeks after transplantation. FIG. 4I shows quantification of NeuN+ neurons, GABA+ neurons, SOX9+ astrocytes, and MBP+ oligodendrocytes among hNu+ human cells at 12 weeks post-transplant (n=6). FIG. 4J shows co-immunostaining for mCherry, GABA, and PTF1A in the graft. Scale bars: 50 μm.

FIG. 5A, FIG. 5B, FIG. 5C, FIG. 5D, FIG. 5E, FIG. 5F, FIG. 5G, FIG. 5H, FIG. 5I, FIG. 5J, FIG. 5K, FIG. 5L, FIG. 5M, FIG. 5N, and FIG. 5O show the localization of and projection of the grafted human neurons. FIG. 5A show the horizontal section cut through the graft site and the caudal (lumbar) white matter, showing the distribution of mCherry+ cells and axons. Inset areas are magnified in FIG. 5B-FIG. 5O. FIG. 5B shows an amplification of inset B, showing mCherry+ cells co-labelled with NeuN. FIG. 5C shows a further magnification of inset B with z stack colocalization. FIG. 5D shows a magnification of inset D to show mCherry positive (human) axons contacting a host NeuN+ neuron. The inset is further magnified in FIG. 5E with z stack colocalization. FIG. 5F shows a magnification of inset F, and FIG. 5G shows a magnification of inset G, to show mCherry positive axons in lumbar white matter. Further shown are overall cross sections of the transplant site showing hNu and mCherry+ (FIG. 5H) and hNu and NeuN+ (FIG. 5I) human cells in the grey matter. FIG. 5J shows a higher magnification and separate channels of the inset shown in FIG. 5I. FIG. 5K shows immunostaining for hSyn and ChAT, showing numerous hSyn punctae on the surface of ChAT+ spinal dl4s. FIG. 5L shows the inset area of FIG. 5K magnified in with Z-section. FIG. 5M is a z stack image showing double-labeling for hSyn (Green) and ChAT (Red). The inset indicates the interested area for EM, and the arrow shows sampling direction of EM sections. FIG. 5N shows results from correlative light and electron microscopy (CLEM), showing multiple hSyn+ ends (green) on the surface of the spinal dl4 (purple). FIG. 5O shows 3D reconstructions of FIB-SEM identify hSyn on MN (n=726 EM sections).

FIG. 6A, FIG. 6B, FIG. 6C, FIG. 6D, FIG. 6E, FIG. 6F, FIG. 6G, and FIG. 6H show the effects of cell transplantation on spasticity. Shown are the mean relative amplitudes of the Hoffmann reflex (H %) in Sham, SCI, SCI+Cell, and SCI+Medium groups at 6 weeks (FIG. 6A) and 12 weeks (FIG. 6B) post-transplantation with an increasing stimulation frequency from 0.1 Hz to 5 Hz (n=6 in each group). Shown in FIG. 6C-FIG. 6H is the effect of CNO treatment on the H % in sham-operated, SCI, SCI+Cell, SCI+Medium groups at 6 weeks (FIG. 6C, FIG. 6D, FIG. 6E) and 12 weeks (FIG. 6F, FIG. 6G, FIG. 6H) post-transplantation with an increasing stimulation frequency from 1 Hz to 5 Hz.

FIG. 7A, FIG. 7B, FIG. 7C, FIG. 7D, FIG. 7E, and FIG. 7F show the effects of cell transplantation on locomotion. FIG. 7A shows scores in sham (black), SCI (red), SCI+Cell (blue), and SCI+Medium (green) groups. FIG. 6B-FIG. 6F show the Stride Time (FIG. 6B), Swing Time (FIG. 6C), Stride Length (FIG. 6D), Stride Frequency (FIG. 6E) of the hindlimbs, and Run Speed (FIG. 6F) in different groups.

FIG. 8A and FIG. 8B show the effects of cell transplantation on pain. FIG. 8A shows the mechanical withdrawal threshold of hindlimbs at 6 weeks postgraft. FIG. 8B shows the mechanical withdrawal threshold of hindlimbs at 12 weeks postgraft.

### DETAILED DESCRIPTION

Described herein are methods and culture media for inducing differentiation of human stem cells to cells that express one or more markers of spinal dl4 progenitors. Further described herein are compositions of cells expressing such markers. The spinal dl4 progenitor cells may be used in methods of treating neurological disorders, such as spinal cord injury.

Developmentally, the inhibitory neurons in the spinal cord are mainly derived from the PTF1A expressing progenitors in the dorsal interneurons domain 4 (dl4). These dl4 progenitors produce both GABAergic and glycinergic neurons that participate in the motor network for coordinating gaits. Following this principle, detailed herein is a strategy to efficiently differentiate human embryonic and induced pluripotent stem cells (ESCs and iPSCs) to dl4 spinal progenitors and subsequently functional GABA interneurons. Upon transplantation into the injured rat spinal cord, the human dl4 progenitors generate GABA neurons and connect with endogenous spinal dl4s, and the transplanted animals exhibit mitigated spasticity, alleviated neuropathic pain, and improved locomotion.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The regulatory elements may include, for example, a promoter, an enhancer, an initiation codon, a stop codon, or a polyadenylation signal. The coding sequence may be codon optimized.

“Complement” or “complementary” as used herein means a nucleic acid can mean Watson-Crick (for example, A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, for example, to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (for example, from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Sample” or “test sample” as used herein can mean any sample in which the presence and/or level of a target is to be detected or determined or any sample comprising a cell or a component of culture medium as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to catalyze a reaction. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Methods of Generating Spinal dl4 Progenitors

Provided herein are methods for generating spinal dl4 progenitors. As used herein, the term “spinal dl4 progenitor” refers to a progenitor or precursor cell that is generated from spinal cord dorsal interneurons domain 4. The spinal dl4 progenitors have a gene expression profile comprising expression of at least one of PTF1A, PAX7, and ASCL1, or a combination thereof. The spinal dl4 progenitors may subsequently generate spinal GABA interneurons. Spinal GABA interneurons have a gene expression profile comprising expression of at least one of PAX2 and LHX1/5 or a combination thereof. Expression of genes as detailed herein, such as PTF1A, PAX7, ASCL1, PAX2, and LHX1/5, may be detected and/or the level determined by any suitable means known in the art. For example, expression may be determined by PCR such as RT-PCR, detection of a labelled probe that is hybridized to mRNA encoding the protein, or detection of an antibody bound to the protein expressed from the gene. Examples of suitable and commercially available antibodies and primer sequences are detailed in TABLE 1 and TABLE 2 in Example 1.

### a. Generating Spinal Neuroepithelia Progenitors in a First Culture Medium

To generate spinal dl4 progenitors, a first step in the method may comprise generating a population of neuroepithelial cells from human stem cells. The human stem cells may be human pluripotent stem cells. The human stem cells may be human embryonic stem cells (hESCs) and/or human induced pluripotent stem cells (hiPSCs). The human stem cells may be obtained from a human embryo. The human stem cells may be cultured in the culture medium for a time sufficient to induce differentiation of the human stem cells into neuroepithelial cells. The human stem cells may be cultured in the culture medium for 5 to 10 days, 5 to 7 days, 7 to 10 days, or 6 to 8 days. The human stem cells may be cultured in the culture medium for at least 5 days, at least 6 days, at least 7 days, less than 8 days, less than 9 days, or less than 10 days. The human stem cells may be cultured in the culture medium for 5 days, 6 days, 7 days, 8 days, 9 days, or 10 days. In some embodiments, the human stem cells are cultured in the culture medium for 7 days to generate neuroepithelial cells.

The population of cells generated from the human stem cells comprise neuroepithelial cells. Neuroepithelial cells are also known as neural stem cells, and the terms “neuroepithelial cell” and “neural stem cell” are used interchangeably herein. The neuroepithelial cells may be spinal neuroepithelia progenitors. The neuroepithelial cells may be caudal neuroepithelia progenitors. Spinal neuroepithelia progenitors may express Sox1, or Hoxa3, or a combination thereof. Spinal neuroepithelia progenitors may express Sox1 and Hoxa3. In some embodiments, at least 80%, at least 85%, at least 90%, at least 95%, or at least 98% of the population of cells generated from the human stem cells are Sox1+/Hoxa3+ spinal neuroepithelia progenitors. As detailed above, expression of genes, such as Sox1 and Hoxa3, may be detected and/or the level determined by any suitable means known in the art. For example, expression may be determined by PCR such as RT-PCR, detection of a labelled probe that is hybridized to mRNA encoding the protein, or detection of an antibody bound to the protein expressed from the gene. Examples of suitable and commercially available antibodies and primer sequences are detailed in TABLE 1 and TABLE 2 in Example 1.

The culture medium for generating a population of cells comprising neuroepithelial cells from human stem cells can comprise a plurality of small molecules or other chemical compounds or components that promote the differentiation of human stem cells into neuroepithelial cells. In some cases, such a culture medium is called a differentiation culture medium. The culture medium may comprise an agonist of the canonical Wnt signaling pathway, an inhibitor of the bone morphogenetic protein (BMP) signaling pathway, and an inhibitor of Activin/Nodal/TGFβ signaling.

The Wingless-INT (Wnt) signaling pathway agonist may comprise a GSK3 inhibitor. The GSK3 inhibitor may comprise CHIR99021, or the Wnt/β-catenin signaling agonist 6-bromo-iridium-3′-oxime (“BIO”; Meijer et al., Chem. Biol. 2003, 10, 1255-1266), or a combination thereof. In some embodiments, the GSK3 inhibitor comprises CHIR99021. By inhibiting GSK3, CHIR99021 activates the canonical Wnt signaling pathway. CHIR99021 has been reported to inhibit the differentiation of mouse and human embryonic stem cells (ESCs) through Wnt signaling (Wray and Hartmann, Trends in Cell Biology 2012, 22, 159-168). CHIR99021 is commercially available from, for example, Selleckchem (Houston, TX; CAS No. 252917-06-9; Catalog No. S1263). In some embodiments, the GSK3 inhibitor comprises the Wnt/β-catenin signaling agonist 6-bromo-iridium-3′-oxime (“BIO”). 6-bromo-iridium-3′-oxime is commercially available, for example, from Selleckchem (Houston, TX; Catalog No. S7198). GSK3 inhibitors such as those described herein are available from commercial vendors of chemical compounds (for example, Selleckchem, Houston, TX; Tocris Bioscience, Bristol, UK). The culture medium may comprise a Wnt signaling pathway agonist in an amount of about 1 μM to about 10 μM, about 0.5 μM to about 15 μM, about 2 μM to about 8 μM, or about 2 μM to about 5 μM. The culture medium may comprise a Wnt signaling pathway agonist in an amount of at least about 1 μM, at least about 2 μM, at least about 3 μM, at least about 4 μM, at least about 5 μM, at least about 6 μM, at least about 7 μM, at least about 8 μM, at least about 9 μM, or at least about 10 μM. The culture medium may comprise a Wnt signaling pathway agonist in an amount of less than about 10 μM, less than about 9 μM, less than about 8 μM, less than about 7 μM, less than about 6 μM, less than about 5 μM, less than about 4 μM, less than about 3 μM, or less than about 2 μM.

The BMP signaling pathway inhibitor may comprise DMH-1, dorsomorphin, or LDN-193189, or a combination thereof. DMH-1 blocks BMP signaling by inhibiting Activin receptor-like kinase (ALK2). Dorsomorphin and LDN-193189 each affect Smad-dependent and Smad-independent BMP signaling triggered by BMP2, BMP6, or GDFS. DMH-1 is commercially available, for example, from Selleckchem (Houston, TX; Catalog No. S7146). Dorsomorphin is commercially available, for example, from Selleckchem (Houston, TX; Catalog No. S7306). LDN-193189 is commercially available, for example, from Selleckchem (Houston, TX; Catalog No. S2618). The culture medium may comprise a BMP signaling pathway inhibitor in an amount of about 1 μM to about 10 μM, about 0.5 μM to about 15 μM, about 1 μM to about 8 μM, or about 1 μM to about 5 μM. The culture medium may comprise a BMP signaling pathway inhibitor in an amount of at least about 0.5 μM, at least about 1 μM, at least about 2 μM, at least about 3 μM, at least about 4 μM, at least about 5 μM, at least about 6 μM, at least about 7 μM, at least about 8 μM, at least about 9 μM, or at least about 10 μM. The culture medium may comprise a BMP signaling pathway inhibitor in an amount of less than about 10 μM, less than about 9 μM, less than about 8 μM, less than about 7 μM, less than about 6 μM, less than about 5 μM, less than about 4 μM, less than about 3 μM, less than about 2 μM, or less than about 1 μM.

The transforming growth factor beta (TGFβ) signaling pathway inhibitor may comprise SB431542, SB505124, or A83-01, or a combination thereof. In some cases, an inhibitor of TGFβ/Activin/Nodal signaling comprises SB431542, which inhibits Activin receptor-like kinases 4, 5, and 7 (ALK4, ALK5, and ALK7). By inhibiting Activin receptor-like kinases 4, 5, and 7, SB431542 inhibits TGFβ/Activin/Nodal signaling. SB505124 and A83-01 are other small molecule inhibitors of Activin receptor-like kinase 5 (ALK5) (also known as transforming growth factor-a type I receptor kinase) that may also be used to inhibit TGFβ/Activin/Nodal signaling. SB431542 can be purchased from any one of several commercial chemical compound vendors (for example, Tocris Bioscience, Bristol, UK; Sigma-Aldrich, St. Louis, MO). For example, SB431542 is commercially available from Selleckchem (Houston, TX; Catalog No. S1067). SB505124 is commercially available, for example, from Selleckchem (Houston, TX; Catalog No. S2186). A83-01 is commercially available, for example, from Selleckchem (Houston, TX; Catalog No. S7692). The culture medium may comprise a TGFβ signaling pathway inhibitor in an amount of about 1 μM to about 10 μM, about 0.5 μM to about 15 μM, about 1 μM to about 8 μM, or about 1 μM to about 5 μM. The culture medium may comprise a TGFβ signaling pathway inhibitor in an amount of at least about 0.5 μM, at least about 1 μM, at least about 2 μM, at least about 3 μM, at least about 4 μM, at least about 5 μM, at least about 6 μM, at least about 7 μM, at least about 8 μM, at least about 9 μM, or at least about 10 μM. The culture medium may comprise a TGFβ signaling pathway inhibitor in an amount of less than about 10 μM, less than about 9 μM, less than about 8 μM, less than about 7 μM, less than about 6 μM, less than about 5 μM, less than about 4 μM, less than about 3 μM, less than about 2 μM, or less than about 1 μM.

In an exemplary embodiment, the culture medium for generating a population of cells comprising neuroepithelial cells from human stem cells comprises the GSK3 inhibitor CHIR99021, the BMP signaling inhibitor DMH-1, and the TGFβ signaling inhibitor SB431542. In some embodiments, the culture medium comprises about 1 μM to about 10 μM CHIR99021, about 1 μM to about 10 μM DMH-1, and about 1 μM to about 10 μM SB431542. In some embodiments, culture medium comprises 2 μM SB431542, 2 μM DMH1, and 3 μM CHIR99021.

In some embodiments, the culture medium for generating a population of cells comprising neuroepithelial cells from human stem cells comprises a culture medium supplemented with a Wnt signaling pathway agonist, a BMP signaling pathway inhibitor, and/or a TGFβ signaling pathway inhibitor, as detailed herein. The conventional culture medium to which a Wnt signaling pathway agonist, a BMP signaling pathway inhibitor, and/or a TGFβ signaling pathway inhibitor may be added may be a neural culture medium. For example, the culture medium may be Neurobasal® culture medium (Life Technologies, Carlsbad, CA). In some cases, the culture medium comprises DMEM/F12, Neurobasal medium at 1:1, 1X N2 neural supplement (N-2 Supplement; Gibco, Dublin, Ireland), 1X B27 neural supplement (B-27 Supplement; Gibco, Dublin, Ireland), and 1 mM ascorbic acid.

Any appropriate culture method can be used with the culture media detailed herein. For example, adherent culture methods can be used. Adherent culture (or “colony culture”) allows direct visualization of neural differentiation, including the formation of neural tube-like rosettes during neuroepithelial induction and the migration of neuroepithelial cells. Adherent/colony culture permits ready removal of non-neural colonies and promotes subsequent neural differentiation. In some cases, suspension culture can be used for initially separating pluripotent cells from mouse embryonic fibroblast (MEF) feeder cells or for purifying neuroepithelial cells.

### b. Generating Spinal dl4 Progenitor Cells in a Second Culture Medium

The method for generating spinal dl4 progenitors can further comprise inducing the neuroepithelial cells (for example, neuroepithelial cells generated as detailed above) to differentiate into spinal dl4 progenitors. The neuroepithelial cells, such as spinal neuroepithelia progenitors, may be cultured in the culture medium for a time sufficient to generate a population of cells comprising spinal dl4 progenitor cells. For example, the neuroepithelial cells, such as spinal neuroepithelia progenitors, may be cultured in the culture medium for a time sufficient to induce expression of a spinal dl4 progenitor marker (such as PTF1A). The neuroepithelial cells may be cultured in the culture medium for 5 to 10 days, 5 to 7 days, 7 to 10 days, or 6 to 8 days. The neuroepithelial cells may be cultured in the culture medium for at least 5 days, at least 6 days, at least 7 days, less than 8 days, less than 9 days, or less than 10 days. The neuroepithelial cells may be cultured in the culture medium for 5 days, 6 days, 7 days, 8 days, 9 days, or 10 days. In some embodiments, the neuroepithelial cells are cultured in the culture medium for 7 days to generate neuroepithelial cells to generate spinal dl4 progenitors.

Spinal dl4 progenitors may be identified on the basis of PTF1A+ expression. The bHLH transcription factor PTF1A serves as a unique marker of spinal dl4 progenitors. The dl4 progenitor fate is dependent on PTF1A and loss of this gene results in loss of all GABAergic dorsal neurons and respecification to dl5 fate (Henke R. et al., Development 2009, 136, 2945-2954; Meredith D. M. et al., J. Neurosci. 2009, 29, 11139-11148). Spinal dl4 progenitor cells may express PTF1A, PAX7, or ASCL1, or any combination thereof. In some embodiments, spinal dl4 progenitor cells express PTF1A, PAX7, and ASCL1. In some embodiments, at least 80%, at least 85%, at least 90%, at least 95%, or at least 98% of the population of cells generated are PTF1A+/PAX7+/ASCL1+ spinal dl4 progenitor cells. As detailed above, expression of genes, such as PTF1A, PAX7, and ASCL1, may be detected and/or the level determined by any suitable means known in the art. For example, expression may be determined by PCR such as RT-PCR, detection of a labelled probe that is hybridized to mRNA encoding the protein, or detection of an antibody bound to the protein expressed from the gene. Examples of suitable and commercially available antibodies and primer sequences are detailed in TABLE 1 and TABLE 2 in Example 1.

In some embodiments, the neuroepithelial cells, such as spinal neuroepithelia progenitors, are cultured a culture medium comprising a Wnt signaling pathway agonist, a BMP signaling pathway inhibitor, and a TGFβ signaling pathway inhibitor, as detailed above, and further comprising a sonic hedgehog (SHH) signaling inhibitor and a retinoic acid (RA) signaling agonist.

The sonic hedgehog (SHH) signaling inhibitor may comprise, for example, cyclopamine. Cyclopamine inhibits SHH signaling by directly targeting Smoothened (“Smo”), a component of the SHH signaling pathway. Other small molecule inhibitors of Smo can be used to inhibit SHH signaling, such as, for example, SANT-1. SHH acts in a graded manner to establish different neural progenitor cell populations (Briscoe et al., Semin. Cell Dev. 1999, 10, 353-362). Cyclopamine is available from several commercial chemical compound vendors (for example, Tocris Bioscience, Bristol, UK; Selleckchem, Houston, TX). For example, cyclopamine is commercially available from Selleckchem (Houston, TX; CAS No. 4449-51-8; Catalog No. S1146). SANT-1 is commercially available, for example, from Selleckchem (Houston, TX; CAS No. 304909-07-7; Catalog No. S7092). The culture medium may comprise a sonic hedgehog (SHH) signaling inhibitor in an amount of about 0.1 μM to about 2 μM, about 0.05 μM to about 5 μM, about 0.1 μM to about 1.0 μM, or about 0.05 μM to about 3 μM. The culture medium may comprise a sonic hedgehog (SHH) signaling inhibitor in an amount of at least about 0.05 μM, at least about 0.1 μM, at least about 0.15 μM, at least about 0.2 μM, at least about 0.25 μM, at least about 0.3 μM, at least about 0.35 μM, at least about 0.4 μM, at least about 0.45 μM, at least about 0.5 μM, at least about 0.55 μM, at least about 0.6 μM, at least about 0.65 μM, at least about 0.7 μM, at least about 0.75 μM, at least about 0.8 μM, at least about 0.85 μM, at least about 0.9 μM, at least about 1.0 μM, at least about 1.5 μM, at least about 2.0 μM, at least about 2.5 μM, at least about 3.0 μM, at least about 3.5 μM, at least about 4.0 μM, at least about 4.5 μM, or at least about 5.0 μM. The culture medium may comprise a sonic hedgehog (SHH) signaling inhibitor in an amount of less than about 5.0 μM, less than about 4.5 μM, less than about 4.0 μM, less than about 3.5 μM, less than about 3.0 μM, less than about 2.5 μM, less than about 2.0 μM, less than about 1.5 μM, less than about 1.0 μM, less than about 0.9 μM, less than about 0.8 μM, less than about 0.7 μM, less than about 0.6 μM, less than about 0.5 μM, less than about 0.4 μM, less than about 0.3 μM, or less than about 0.2 μM.

The retinoic acid (RA) signaling agonist may include, for example, retinoic acid (RA), or EC23, or a combination thereof. In some embodiments, the RA is all-trans retinoic acid. RA activates RA signaling by binding nuclear hormone receptors retinoic acid receptors (RARs), which is required for specification of spinal dl4 progenitors (Novitch et al., Neuron 2003, 40, 81-95). RA is available from several commercial chemical compound vendors (for example, Tocris Bioscience, Bristol, UK; Sigma-Aldrich, St. Louis, MO). For example, RA is commercially available from Tocris Bioscience (Bristol, UK; Catalog No. 0695). EC23 is commercially available, for example, from Tocris Bioscience (Bristol, UK; Cat. No. 4011). The culture medium may comprise a retinoic acid (RA) signaling agonist in an amount of about 0.01 μM to about 2 μM, about 0.005 μM to about 5 μM, about 0.01 μM to about 1.0 μM, or about 0.05 μM to about 3 μM. The culture medium may comprise a retinoic acid (RA) signaling agonist in an amount of at least about 0.005 μM, at least about 0.01 μM, at least about 0.02 μM, at least about 0.03 μM, at least about 0.04 μM, at least about 0.05 μM, at least about 0.06 μM, at least about 0.07 μM, at least about 0.08 μM, at least about 0.09 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, least about 0.9 μM, at least about 1.0 μM, at least about 1.1 μM, at least about 1.2 μM, at least about 1.3 μM, at least about 1.4 μM, at least about 1.5 μM, at least about 1.6 μM, at least about 1.7 μM, at least about 1.8 μM, at least about 1.9 μM, or at least about 2.0 μM. The culture medium may comprise a retinoic acid (RA) signaling agonist in an amount of less than about 2.0 μM, less than about 1.5 μM, less than about 1.0 μM, less than about 0.9 μM, less than about 0.8 μM, less than about 0.7 μM, less than about 0.6 μM, less than about 0.5 μM, less than about 0.4 μM, less than about 0.3 μM, less than about 0.2 μM, less than about 0.1 μM, less than about 0.09 μM, less than about 0.08 μM, less than about 0.07 μM, less than about 0.06 μM, less than about 0.05 μM, less than about 0.04 μM, less than about 0.03 μM, or less than about 0.02 μM.

In an exemplary embodiment, the culture medium for generating spinal dl4 progenitors from spinal neuroepithelia progenitors comprises Wnt signaling agonist CHIR99021, BMP signaling inhibitor DMH-1, TGFβ signaling inhibitor SB431542, SHH signaling inhibitor cyclopamine, and retinoic acid. In some cases, the culture medium comprises about 1 μM to about 10 μM CHIR99021, about 1 μM to about 10 μM DMH-1, about 1 μM to about 10 μM SB431542, about 0.1 μM to about 2 μM cyclopamine, and about 0.01 μM to about 2 μM RA. In some embodiments, the culture medium comprises 2 μM SB431542, 2 μM DMH1, 3 μM CHIR99021, 0.1 μM all-trans retinoic acid, and 0.5 μM cyclopamine.

Any appropriate culture method can be used with the culture media detailed herein. For example, adherent culture methods can be used.

In some embodiments, at least a portion of the spinal dl4 progenitor cells further differentiate into spinal GABA interneurons. In some cases, the method further includes culturing the spinal dl4 progenitors in a culture medium to generate GABAergic interneuron. The spinal GABA interneurons may express at least one gene selected from PAX2 and LHX1/5, or a combination thereof. For example, the spinal dl4 progenitors may be cultured in a neuron differentiation culture medium to generate PAX2+, LHX1/5+ GABAergic interneurons. Lhx1 and Lhx5 maintain the inhibitory-neurotransmitter status of interneurons in the dorsal spinal cord (Pillai A. et al., Development 2007, 134, 357-366). As detailed above, expression of genes, such as PAX2, and LHX1/5, may be detected and/or the level determined by any suitable means known in the art. For example, expression may be determined by PCR such as RT-PCR, detection of a labelled probe that is hybridized to mRNA encoding the protein, or detection of an antibody bound to the protein expressed from the gene. Examples of suitable and commercially available antibodies and primer sequences are detailed in TABLE 1 and TABLE 2 in Example 1. The spinal dl4 progenitors may be cultured in a neuron differentiation culture medium for 5 to 10 days, 5 to 7 days, 7 to 10 days, or 6 to 8 days. The spinal dl4 progenitors may be cultured in a neuron differentiation culture medium for at least 5 days, at least 6 days, at least 7 days, less than 8 days, less than 9 days, or less than 10 days. The spinal dl4 progenitors may be cultured in a neuron differentiation culture medium for 5 days, 6 days, 7 days, 8 days, 9 days, or 10 days. In some embodiments, the spinal dl4 progenitors are cultured in a neuron differentiation culture medium for approximately one week. In some embodiments, the spinal dl4 progenitors are cultured in a neuron differentiation culture medium for 7 days.

### 3. Method of Treating Neurological Disorders

Further provided herein are methods for treating a neurological disorder in a subject. The in vitro differentiated cells that express one or more markers of a spinal GABA neuron, or spinal dl4 progenitors thereof (also referred to as “stem-cell-derived spinal dl4 progenitors”) as detailed herein, can be used for treating a neurological disorder. The methods may include administering an effective amount of the stem-cell-derived spinal dl4 progenitors, as detailed herein, into a subject suffering from a neurological disorder. In some embodiments, the stem-cell-derived spinal dl4 progenitors are differentiated stem-cell-derived spinal dl4 progenitors. In some embodiments, a portion of the spinal dl4 progenitor cells have further differentiated into spinal GABA interneurons prior to administration. In some embodiments, a portion of the spinal dl4 progenitor cells further differentiate into spinal GABA interneurons after administration. The methods may include administering an effective amount of spinal GABA interneurons, as detailed herein, to a subject suffering from a neurological disorder. The administered or transplanted cells may exhibit a therapeutic effect to mitigate spasticity, alleviate neuropathic pain, and/or improve locomotion in the subject.

Non-limiting examples of a neurological disorders include spinal cord injury. In some embodiments, the neurological disorder comprises a spinal cord injury. A spinal cord injury (SCI) is damage to the spinal cord that results in a loss of function, such as mobility and/or feeling. Frequent causes of spinal cord injuries are trauma (car accident, gunshot, falls, etc.) or disease (polio, spina bifida, Friedreich's ataxia, etc.). Treatment of a SCI may result in spinal cord nerve regeneration, recovery of nerve functioning and behavior, or a combination thereof. Spinal cord injuries may include both acute and chronic spinal cord injuries. The term “acute injury” includes injuries that have recently occurred. For example, an acute injury may have very recently occurred, may have occurred within an hour or less, may have occurred within a day or less, may have occurred within a week or less, or may have occurred within two weeks or less. The term “chronic injury” is an injury that has persisted for a period of time. For example, a chronic injury may have occurred more than two weeks ago, may have occurred more than three weeks ago, may have occurred more than two months ago, or may have occurred more than three months ago. The term “injury” generally denotes a breakdown of the membrane of a nerve cell, such that there is a collapse in the ability of the nerve membrane to separate the salty gel on their insides (cytoplasm) from the salty fluid bathing them (extracellular fluid). The types of salts in these two fluid compartments are very different and the exchange of ions and water caused by injury leads to the inability of the nerve to produce and propagate nerve impulses—and further to the death of the cell. An SCI may include damage that directly or indirectly affects the normal functioning of the central nervous system (CNS). The SCI may be a structural, physical, or mechanical impairment and may be caused by physical impact, as in the case of a crushing, compression, or stretching of nerve fibers. Alternatively, the cell membrane may be destroyed by or degraded by an illness, a chemical imbalance, or a physiological malfunction such as anoxia (for example, stroke), aneurysm, or reperfusion. A CNS injury includes, for example and without limitation, damage to retinal ganglion cells, a traumatic brain injury, a stroke-related injury, a cerebral aneurism-related injury, a spinal cord injury, including monoplegia, diplegia, paraplegia, hemiplegia and quadriplegia, a neuroproliferative disorder, or neuropathic pain syndrome. With injury to the spinal cord of a mammal, connections between nerves in the spinal cord may be broken. Such injuries block the flow of nerve impulses for the nerve tracts affected by the injury, with a resulting impairment to both sensory and motor function. Injuries to the spinal cord may arise from compression or other contusion of the spinal cord, or a crushing or severing of the spinal cord. A severing of the spinal cord, also referred to herein as a “transection,” may be a complete severing or may be an incomplete severing of the spinal cord.

The spinal neuroepithelia progenitors and/or spinal dl4 progenitor cells, or pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed cells or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof.

The presently disclosed stem-cell-derived spinal dl4 progenitors can be administered or provided systemically or directly to a subject for treating or preventing a neurological disorder. In certain embodiments, the presently disclosed stem-cell-derived spinal dl4 progenitors are directly injected into an organ of interest (for example, the central nervous system (CNS) or peripheral nervous system (PNS)). In certain embodiments, the presently disclosed stem-cell-derived spinal dl4 progenitors are directly injected into the spinal cord. The spinal neuroepithelia progenitors and/or spinal dl4 progenitor cells, or pharmaceutical compositions comprising the same, may be injected into the brain or other component of the central nervous system.

Pharmaceutical compositions comprising the above-described spinal neuroepithelia progenitors and/or spinal dl4 progenitor cells may be formulated in accordance with standard techniques well known to those skilled in the pharmaceutical art. The pharmaceutical compositions can be formulated according to the mode of administration to be used. The pharmaceutical composition may comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof.

The stem-cell-derived spinal dl4 progenitors can be administered in any physiologically acceptable vehicle. Pharmaceutical compositions comprising the spinal dl4 progenitors and a pharmaceutically acceptable vehicle are also provided. The spinal dl4 progenitors and the pharmaceutical compositions comprising said cells can be administered via localized injection, orthotopic (OT) injection, systemic injection, intravenous injection, or parenteral administration. In certain embodiments, the spinal dl4 progenitors are administered to a subject suffering from a neurological disorder via orthotopic (OT) injection.

The stem-cell-derived spinal dl4 progenitors and the pharmaceutical compositions comprising said cells can be conveniently provided as sterile liquid preparations, for example, isotonic aqueous solutions, suspensions, emulsions, dispersions, or viscous compositions, which may be buffered to a selected pH. Liquid preparations may be easier to prepare than gels, other viscous compositions, and solid compositions. Additionally, liquid compositions may be more convenient to administer, especially by injection. Viscous compositions can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, for example, a composition comprising the presently disclosed stem-cell-derived precursors, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can be lyophilized. The compositions can contain auxiliary substances such as wetting, dispersing, or emulsifying agents (for example, methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives that enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the presently disclosed subject matter, however, any vehicle, diluent, or additive used would have to be compatible with the presently disclosed stem-cell-derived pregenitors.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. An amount may be used to achieve the selected viscosity. The choice of suitable carriers and other additives will depend on the exact route of administration and the nature of the particular dosage form, for example, liquid dosage form (for example, whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the spinal dl4 progenitors. This will present no problem to those skilled in chemical and pharmaceutical principles, or problems can be readily avoided by reference to standard texts or by simple experiments (not involving undue experimentation), from this disclosure and the documents cited herein.

In certain non-limiting embodiments, the spinal dl4 progenitors described herein are comprised in a composition that further comprises a biocompatible scaffold or matrix, for example, a biocompatible three-dimensional scaffold that facilitates tissue regeneration when the cells are implanted or grafted to a subject. In certain non-limiting embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel (see, for example, U.S. Publication Nos. 2015/0159135, 2011/0296542, 2009/0123433, and 2008/0268019, the contents of each of which are incorporated by reference in their entireties). In certain embodiments, the composition further comprises growth factors for promoting maturation of the implanted/grafted cells into spinal GABA interneurons.

One consideration concerning the therapeutic use of the spinal dl4 progenitors is the quantity of cells necessary to achieve an optimal effect. An optimal effect includes, but is not limited to, repopulation of CNS and/or PNS regions of a subject suffering from a neurological disorder, and/or improved function of the subject's CNS and/or PNS.

An “effective amount” (or “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the neurological disorder or otherwise reduce the pathological consequences of the neurological disorder. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the spinal dl4 progenitors is an amount that is sufficient to repopulate CNS and/or PNS regions of a subject suffering from a neurological disorder. In certain embodiments, an effective amount of the spinal dl4 progenitors is an amount that is sufficient to improve the function of spinal cord of a subject, such as to mitigate spasticity, alleviate neuropathic pain and improve locomotion, for example, the improved function can be about 1%, about 5%, about 10%, about 20%, about 30%, about 40%, about 50%, about 60%, about 70%, about 80%, about 90%, about 95%, about 98%, about 99% or about 100% of the function of a normal person's spinal cord.

The quantity of cells to be administered will vary for the subject being treated. The precise determination of what would be considered an effective dose may be based on factors individual to each subject, including their size, age, sex, weight, and condition of the particular subject. Dosages can be readily ascertained by those skilled in the art from this disclosure and the knowledge in the art.

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims

### 4. Examples

### Example 1

### Efficient Generation of Spinal dl4 Progenitors from Human ESCs in 2 Weeks

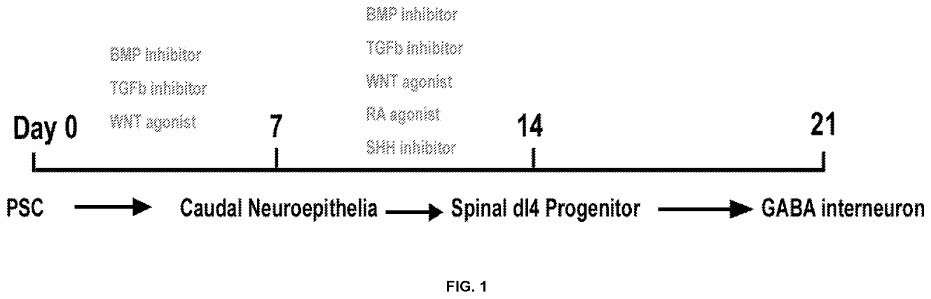

To induce the specification of neuroepithelial cells from human pluripotent cells, the dual TGFβ/BMP inhibition approach was applied for human embryonic stem cells in a monolayer culture (see, for example, Chambers et al., Nature Biotech. 2009, 27, 275-280). FIG. 1 is a flow chart depicting directed differentiation of spinal dl4 progenitors and GABA interneurons from pluripotent stem cells. The small molecule SB431542 represses TGFβ signaling by selectively inhibiting Activin receptor-like kinase ALK4/5/7. The small molecule DMH-1 represses BMP signaling by selectively inhibiting the BMP receptor kinase ALK2. Human embryonic stem cells (hESCs) were treated in cell culture with 2 μM DMH-1 and 2 μM SB431542 for 1 week. The cell culture medium was a neural culture medium including DMEM/F12, Neurobasal medium at 1:1, 1X N2 neural supplement (N-2 Supplement; Gibco, Dublin, Ireland), 1X B27 neural supplement (B-27 Supplement; Gibco, Dublin, Ireland), and 1 mM ascorbic acid (along with the 2 μM DMH-1 and 2 μM SB431542). The cells were cultured at regular conditions of 5% CO2 and 37° C. Treated hESCs were then induced to differentiate into populations comprising about 85% SOX1+ neuroepithelial cells but also comprising other cell lineages due to spontaneous ESC differentiation, since the dual TGFβ/BMP inhibitors SB431542 and DMH-1 are unable to prevent all spontaneous differentiation into other cell lineages, especially when ESC colonies are small. A small molecule that inhibits glycogen synthase kinase-3 (CHIR99021) was used to maintain the ESC state during culturing. The antibodies used in these experiments are detailed in TABLE 1. Primers for PCR are detailed in TABLE 2.

TABLE 1
List of antibodies.

| REAGENT OR RESOURCE | SOURCE | IDENTIFIER |
|---|---|---|
| Mouse monoclonal anti-ASCL1 | Santa Cruz Biotechnology | sc-374104, RRID: |
| | (Dallas, TX) | AB_10918561 |
| Goat polyclonal anti-ChAT | Millipore (Burlington, MA) | AB144P, RRID: |
| | | AB_2079751 |
| Rabbit polyclonal | Abcam (Cambridge, UK) | ab18723, RRID: |
| anti-Doublecortin (DCX) | | AB_732011 |
| Rabbit polyclonal anti-FOXG1 | Thermo Fisher (Waltham, MA) | PA5-41493, RRID: |
| | | AB_2608491 |
| Rabbit polyclonal anti-GABA | Sigma-Aldrich (St. Louis, MO) | A2052, RRID: |
| | | AB_477652 |
| Mouse monoclonal anti-GABA | Sigma-Aldrich (St. Louis, MO) | A0310, RRID: |
| | | AB_476667 |
| Mouse monoclonal anti-GAD65 | Millipore (Burlington, MA) | MAB351, RRID: |
| | | AB_11214081 |
| Mouse monoclonal anti-GAD65/67 | Santa Cruz Biotechnology | sc-365180, RRID: |
| | (Dallas, TX) | AB_10710523 |
| Rabbit polyclonal anti-HOXA3 | Sigma-Aldrich (St. Louis, MO) | HPA029157, RRID: |
| | | AB_10601020 |
| Rat monoclonal anti-HOXB4 | DSHB (University of Iowa) | I12 anti-Hoxb4, |
| | | RRID: AB_2119288 |
| Mouse monoclonal anti-Lhx1/5 | DSHB (University of Iowa) | 4F2, RRID: AB_531784 |
| Mouse monoclonal | Millipore (Burlington, MA) | MAB1281, RRID: |
| anti-humanNuclei (hNu) | | AB_94090 |
| Rabbit polyclonal anti-Iba1 | Wako (Richmond, VA) | 019-19741, RRID: |
| | | AB_839504 |
| Rabbit polyclonal anti-Ki67 | Abcam (Cambridge, UK) | ab15580, RRID: |
| | | AB_443209 |
| Mouse monoclonal anti-MAP2 | Sigma-Aldrich (St. Louis, MO) | M1406, RRID: |
| | | AB_477171 |
| Rabbit polyclonal anti-Myelin | Abcam (Cambridge, UK) | ab40390, RRID: |
| Basic Protein (MBP) | | AB_1141521 |
| Chicken polyclonal anti-mCherry | Novus (Littleton, CO) | NBP2-25158, RRID: |
| | | AB_2636881 |
| Rabbit polyclonal anti-mCherry | Abcam (Cambridge, UK) | ab167453, RRID: |
| | | AB_2571870 |
| Goat polyclonal anti-mCherry | Biorbyt (Cambridge, UK) | Cat# orb11618, |
| | | RRID: AB_2687829 |
| Mouse monoclonal anti-NeuN | Millipore (Burlington, MA) | MAB377, RRID: |
| | | AB_2298772 |
| Rabbit polyclonal | Sigma-Aldrich (St. Louis, MO) | N4142, RRID: |
| anti-Neurofilament 200 (NF-200) | | AB_477272 |
| Rabbit polyclonal anti-Olig-2 | Millipore (Burlington, MA) | AB9610, RRID: |
| | | AB_570666 |
| Rabbit polyclonal anti-Pax2 | BioLegend (San Diego, CA) | 901001, RRID: |
| | | AB_2565001 |
| Mouse monoclonal anti-Pax7 | R&DSYSTEMS (Minneapolis, MN) | MAB1675, RRID: |
| | | AB_2159833 |
| Mouse monoclonal anti PTF1A | BD Biosciences (Franklin Lakes, NJ) | 564745, RRID: |
| | | AB_2738928 |
| Goat polyclonal anti-SOX1 | R&DSYSTEMS (Minneapolis, MN) | AF3369, RRID: |
| | | AB_2239879 |
| Goat polyclonal anti-SOX9 | R&DSYSTEMS (Minneapolis, MN) | AF3075, RRID: |
| | | AB_2194160 |
| Mouse monoclonal anti-STEM121 | Takara Bio (Kusatsu, Shiga, Japan) | Y40410, RRID: |
| | | AB_2801314 |
| Mouse monoclonal anti-Synaptophysin | Millipore (Burlington, MA) | MAB329, RRID: |
| | | AB_94786 |
| Mouse monoclonal anti-human | Thermo Fisher (Waltham, MA) | 14-6525-80, RRID: |
| Synaptophysin (hSyn) | | AB_10670424 |
| Rabbit polyclonal | BioLegend (San Diego, CA) | 802001, RRID: |
| anti-Tubulin beta-3 | | AB_2564645 |
| Rabbit polyclonal anti-VGLUT 1 | Synaptic Systems (Germany) | 135 302, RRID: |
| | | AB_887877 |
| Mouse monoclonal anti-VGLUT 2 | Millipore (Burlington, MA) | MAB5504, RRID: |
| | | AB_2187552 |
| Donkey anti-Rabbit IgG | Thermo Fisher (Waltham, MA) | A-21206, RRID: |
| Alexa Fluor 488 | | AB_2535792 |
| Donkey anti-Mouse IgG | Thermo Fisher (Waltham, MA) | A-21202, RRID: |
| Alexa Fluor 488 | | AB_141607 |
| Donkey anti-Rat IgG | Thermo Fisher (Waltham, MA) | A-21208, RRID: |
| Alexa Fluor 488 | | AB_2535794 |
| Donkey anti-Mouse IgG | Thermo Fisher (Waltham, MA) | A-21203, RRID: |
| Alexa Fluor 594 | | AB_2535789 |
| Donkey Anti-Chicken IgY (IgG) Cy3 | Jackson ImmunoResearch | 703-165-155, RRID: |
| | (West Grove, PA) | AB_2340363 |
| Donkey Anti-Rabbit IgG Cy5 | Jackson ImmunoResearch | 711-175-152, RRID: |
| | (West Grove, PA) | AB_2340607 |
| Donkey Anti-Mouse IgG Cy5 | Jackson ImmunoResearch | 715-175-151, RRID: |
| | (West Grove, PA) | AB_2340820 |
| Donkey anti-Rabbit IgG | Thermo Fisher (Waltham, MA) | Cat# A-31573, RRID: |
| Alexa Fluor 647 | | AB_2536183 |

TABLE 2
Primer sequences.

| GENE | FORWARD PRIMER SEQUENCE | REVERSE PRIMER SEQUENCE |
|---|---|---|
| BHLHB5 | GCCTGCCTCTCCGCTCAC | ATCTCTTCGTCTTCCTCGTCCTC |
| | (SEQ ID NO: 1) | (SEQ ID NO: 2) |
| BRn3A | GCTGAATCTCCAAGCCTCGT | TGTTTTCGCCCAACATGCAG |
| | (SEQ ID NO: 3) | (SEQ ID NO: 4) |
| DBX2 | ACGGTCTGCTCTGGGTTTCC | ATCTTGGACTTGAGTGCTGTTGG |
| | (SEQ ID NO: 5) | (SEQ ID NO: 6) |
| EN1 | GGCGTCAACAACCTCACTGG | TGAACCTGTCCTTTGTGTATCTGC |
| | (SEQ ID NO: 7) | (SEQ ID NO: 8) |
| ISL-1 | TGCCCGCTCCAAGGTGTATC | GAGACCGTCCTCCCGAAGC |
| | (SEQ ID NO: 9) | (SEQ ID NO: 10) |
| LBX-1 | GCCTGCCTCTCCGCTCAC | ATCTCTTCGTCTTCCTCGTCCTC |
| | (SEQ ID NO: 11) | (SEQ ID NO: 12) |
| LMX1B | CCAAATGCCAGGGAACGACTC | GTCTGAGGAGCCGAGGAAGC |
| | (SEQ ID NO: 13) | (SEQ ID NO: 14) |
| Math1 | AGAGTGGGCTGAAGTGAAGGAG | CGGTTGCGGGAGATGATGC |
| | (SEQ ID NO: 15) | (SEQ ID NO: 16) |
| NGN1 | GCGCCTTTCTATCTGTCCGT | CACAGTCTTCCTCGTCGGTG |
| | (SEQ ID NO: 17) | (SEQ ID NO: 18) |
| NGN2 | GGCAGGTGTAGCCTTTCTGA | TCAGTCCGCTCTGCAAACTC |
| | (SEQ ID NO: 19) | (SEQ ID NO: 20) |
| OLIG3 | ACGGTCTGCTCTGGGTTTCC | ATCTTGGACTTGAGTGCTGTTGG |
| | (SEQ ID NO: 21) | (SEQ ID NO: 22) |
| PAX2 | ATCTGCATCCACCAACCCTG | GCTGAATCTCCAAGCCTCGT |
| | (SEQ ID NO: 23) | (SEQ ID NO: 24) |
| TLX3 | AGCCTCAACGACTCCATCCAG | GTGACAGCGGGAACCTTGG |
| | (SEQ ID NO: 25) | (SEQ ID NO: 26) |
| WT1 | CACACAACGCCCATCCTCTG | CCTGAATGCCTCTGAAGACACC |
| | (SEQ ID NO: 27) | (SEQ ID NO: 28) |

To further improve neural specification, a small molecule that inhibits glycogen synthase kinase-3 (CHIR99021) was applied to the cell culture in combination with DMH-1 and SB431542. GSK3 negatively regulates WNT signaling, and WNT signaling promotes the self-renewal of ESCs and neural progenitors. When exposed to these three molecules for about 7 days, hESCs not only generated more pure populations of SOX1+ neuroepithelial cells (for example, at least 95% of cells in the total population were SOX1+ neuroepithelial cells), but also generated 2.5-fold more neuroepithelial cells. However, CDS (“CDS” stands for CHIR99021, DMH-1, and SB431542) treatment-derived neuroepithelial cells showed caudal identity as demonstrated by staining for HOXA3. By contrast, DS (“DS” stands for DMH-1 and SB431542) treatment-derived neuroepithelial cells showed rostral identity as demonstrated by staining for OTX2.

The efficiency of spinal dl4 progenitor generation from these two populations of neuroepithelial cells was then compared. After treatment with 0.1 μM Retinoic Acid (RA) and 0.5 μM cyclopamine (a small molecule for inhibiting SHH signaling) for another 7 days, more than 90% PTF1A+ spinal dl4 progenitors were induced from CDS treatment-derived neuroepithelial cells, but only 60% from DS treatment-derived neuroepithelial cells. These data suggest an efficient approach for inducing spinal dl4 progenitors from pluripotent stem cells by contacting the stem cells with a three-molecule cocktail of CDS (CHIR99021, DMH-1, and SB431542) or a cocktail of compounds affecting the Wnt pathway, the BMP pathway, and the Activin/Nodal signaling pathway respectively.

### Example 2

### Human Spinal dl4 Progenitors Alleviate Spasticity, Pain and Improve Locomotion in Rats with Spinal Cord Injury

### Spinal GABA interneurons are Efficiently Generated from hPSCs

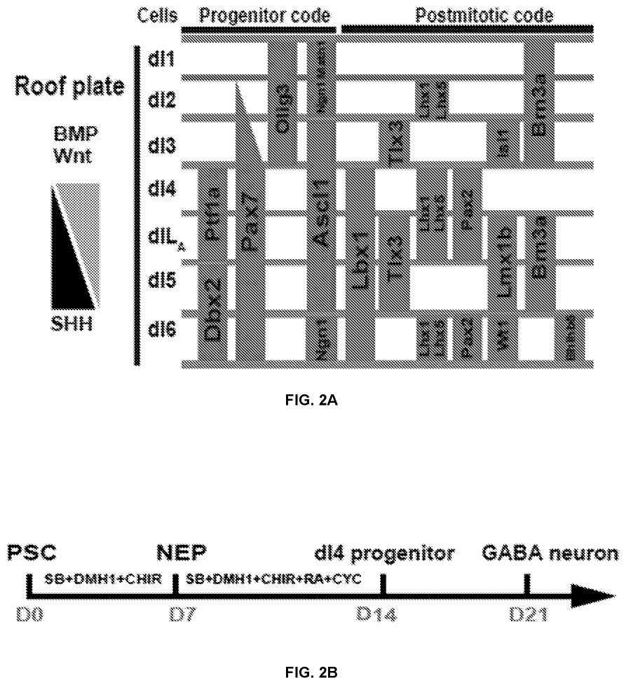

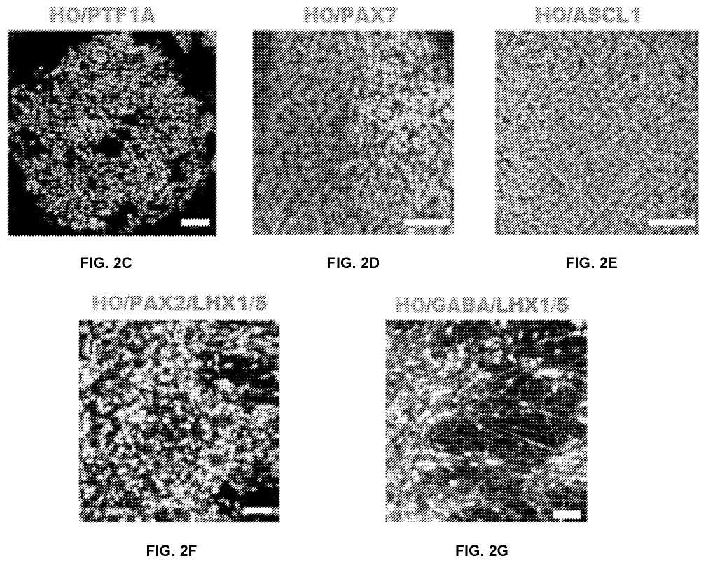

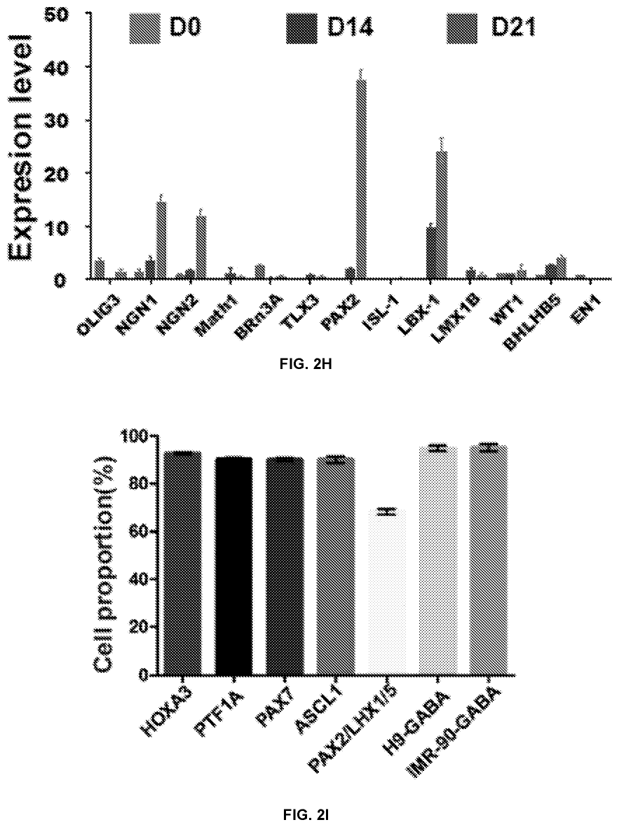

The spinal dl4 progenitors generated according to Example 1 were further examined. The spinal inhibitory neurons originated from the dl4 domain of the developing spinal cord (FIG. 2A) (Lai et al. Development 2016, 143, 3434-3448). We devised a protocol to first induce neuroepithelia from the hESCs (Du et al. Nat. Commun. 2015, 6, 662) and then patterned the neuroepithelia to progenitors in the dl4 domain of the spinal cord (FIG. 2A and FIG. 2B). In the presence of SB431542 (2 μM), DMH1 (2 μM), and CHIR99021 (3 μM) for 7 days, the hESCs were differentiated into a nearly uniform population of SOX1− expressing neuroepithelia organized in rosette forms. In the presence of retinoic acid (RA, 0.1 μM) and cyclopamine (0.5 μM), a sonic hedgehog antagonist, from day 7-14, the neuroepithelia were differentiated to dorsal spinal progenitors, as evidenced by expression of HOXA3 and HOXB4, homeodomain transcription factors expressed in the hindbrain and spinal cord, in more than 90% of the differentiated cells. Analysis of dorsal-ventral markers showed that more than 90% of cells were PTF1A+ at day 14 (FIG. 2C and FIG. 2I), and 90% of the cells were positive for dorsal transcription factor PAX7 and ASCL1 (FIG. 2D, FIG. 2E, and FIG. 2I). PTF1A, PAX7, and PAX2 together with LHX1/5+ were relatively restricted to dl4 progenitors in the spinal cord (FIG. 2A). Correspondingly, no cells were found positive for OLIG2, a ventral marker, or for FOXG1, a forebrain marker. RT-PCR analysis showed that these cells expressed additional dl4-related dorsal transcription factor genes, including LBX-1 and PAX2, but relatively low or no expression of dorsal dl1-3 associated genes BRN3A, MATH1, TLX3, and ISL-1; dl5-6 related transcription factor genes LMX1B, WT1, TLX3, and BHLHB5; and the V1 regional gene EN1 (FIG. 2H). These results indicated that the majority of the differentiated cells were dorsal spinal dl4 progenitors.

Following the withdrawal of morphogens, the spinal progenitors were further differentiated to neurons, by culturing in neural culture medium comprising DMEM/F12, Neurobasal medium at 1:1, 1X N2 neural supplement (N-2 Supplement; Gibco, Dublin, Ireland), 1X B27 neural supplement (B-27 Supplement; Gibco, Dublin, Ireland), and 1 mM ascorbic acid, and also with neural trophic factors BDNF, GDNF, and TGFb. By day 21, the majority of cells (93.2%±3.5%) were process-bearing neurons, as revealed by positive immunostaining for NF-200 in more than 90% of the GABA+ cells. Many of the GABA neurons retained the expression of PAX2 and LHX1/5 (FIG. 2F and FIG. 2G). Since dl4 progenitors can also give rise to glycine neurons, we examined the cultures and did not find cells that were positive for GlyT1+ even at 50 days post-differentiation. Immunostaining for VGIuT2+ showed 2.5%±0.8% positive cells, suggesting the relative absence of glutamate neurons under this differentiation scheme. Thus, the differentiated cells were primarily spinal GABA interneurons.

To validate if the differentiation scheme applies to other cell lines, we differentiated iPSCs (IMR-90 line) to spinal GABA neurons using the same protocol. About 95%±1.82% of the differentiated cells were positive for GABA, similar to that from H9 ESCs (FIG. 2I). Thus, both human ESCs and iPSCs were efficiently directed to spinal somatosensory GABA neurons that bear characteristics of the dl4 regional identity.

### The Spinal GABA Neurons Possess the Characteristic Firing Patterns

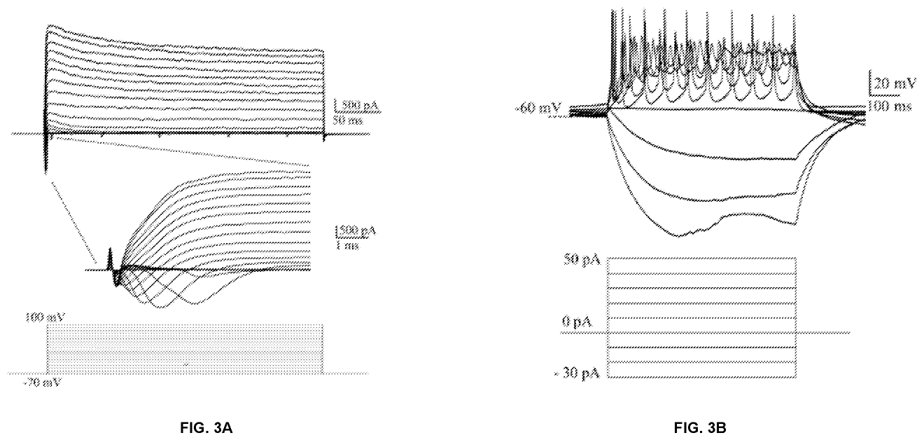

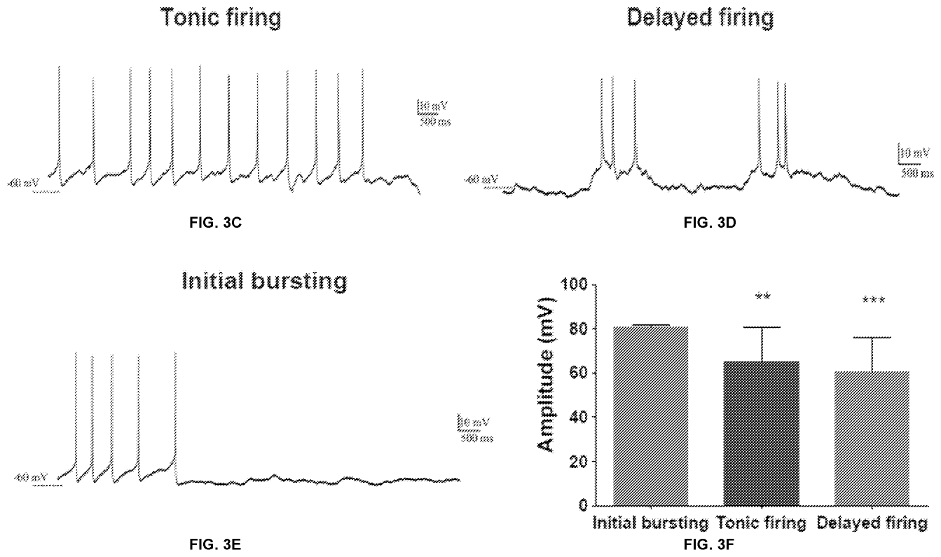

In rodents, the spinal somatosensory neurons possess characteristic firing patterns, mostly exhibiting a tonic firing pattern. Tonic firing typically occurs without presynaptic input and is often characterized by a steady action potential firing at a constant frequency. By patch-clamping electrophysiological recording on the differentiated cells at day 40, we found that the outward K+ and inward Na+ currents were evoked by electrical stimulation from −70 mV to +100 mV at 10 mV increments in most of the cells (FIG. 3A). Action potentials were evoked by current steps from −30 pA to +50 pA in 10 pA increments (FIG. 3B). Many neurons also showed spontaneous action potentials (FIG. 3C-FIG. 3E). Among the 30 neurons patched, 16 displayed a tonic firing pattern with a frequency of 1.6 Hz±0.29 Hz (FIG. 3C), similar to that of the dorsal horn GABA neurons in mice. Six showed a delayed firing pattern (FIG. 3D) and few initial bursting (2 out of 30, FIG. 3E). The amplitude of tonic firing and delayed firing was smaller than that of the initial bursting (FIG. 3F). Therefore, the majority of the in vitro generated neurons possessed the characteristic firing pattern of spinal inhibitory neurons.

### Transplanted Human Spinal dl4 Progenitors in the Injured Spinal Cord

The spinal GABA neurons play a role in regulating spinal dl4 excitability by synapsing with spinal dl4s. Loss of these GABA neurons, as in SCI, is a main pathological basis underlying spasticity (over excitation of spinal dl4s). To determine if our in vitro generated spinal GABA neurons can mitigate spasticity and improve locomotion in SCI, we created a SCI model in rats at the T11/12 spinal cord by a 10 g impactor falling from 25-mm height onto the exposed spinal cord after laminectomy at T10 vertebra. The impacted rats presented bilateral hindlimb twitching and tail flicking and the injured spinal cord appeared swollen. This SCI model allows for assessment of spasticity. The animals that underwent only laminectomy without impact onto the spinal cord served as sham controls.

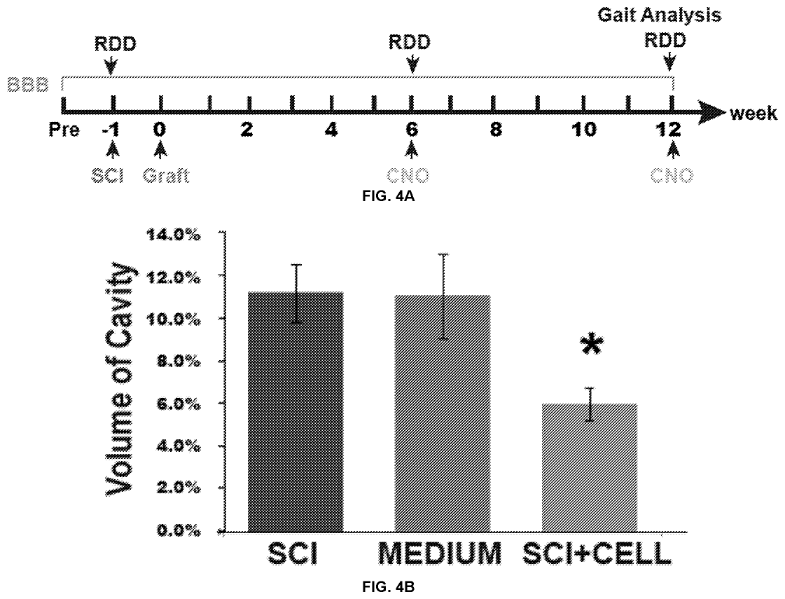

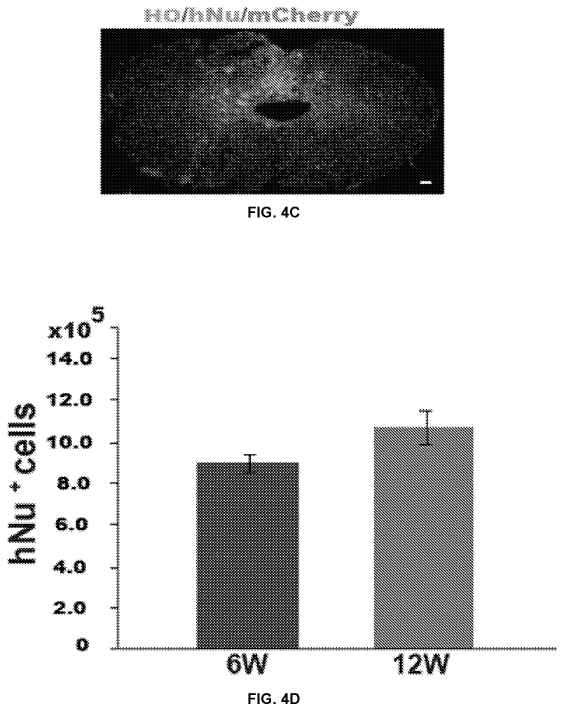

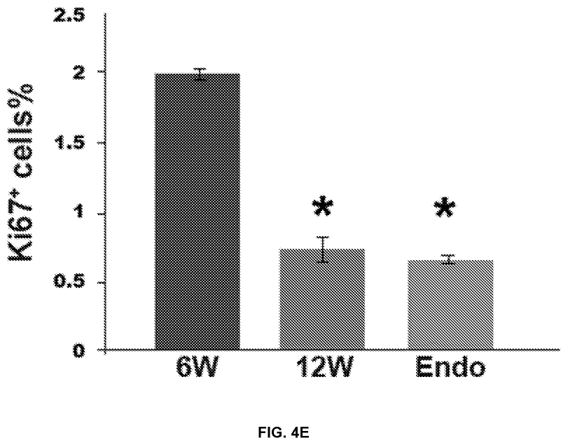

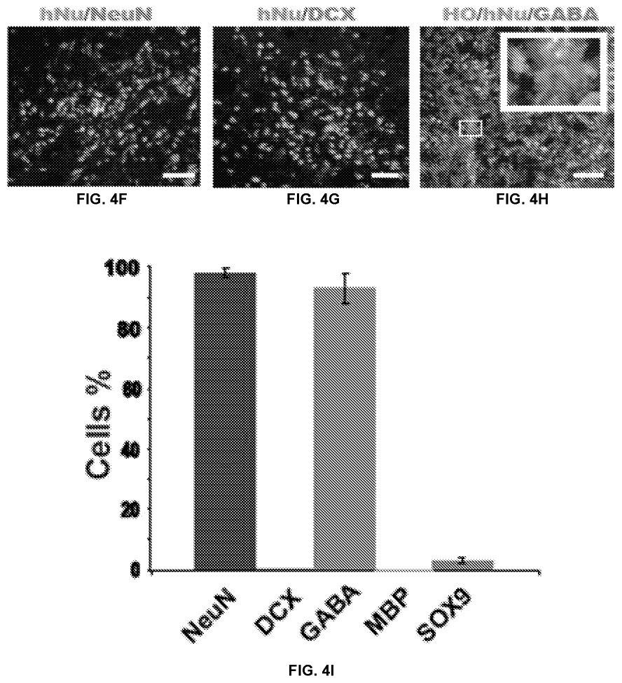

One week after injury, we transplanted 10⁶ (in 3 μL medium consisting of neural basal medium supplemented with B27) hESC-derived dl4 progenitors into the spinal cord at the border of T10/T11 (FIG. 4A). Eighty rats were randomly divided into 4 groups, sham group (laminectomy without SCI), SCI group, SCI with cell transplantation group, and SCI with medium injection

Six and 12 weeks after transplantation, the animals were analyzed histologically. The injured spinal cord showed shrunk at the injury site. HE staining showed a cavity in the injured spinal cord with disorganized tissues. The cavity size was 5.93%±0.79% of the spinal cord sections in the SCI+Cell group but 11.14%±1.3% and 10.95%±1.96% in the SCI group and SCI+Medium group at 12 weeks, respectively (FIG. 4B, SCI+Cell vs. SCI, p=0.02, SCI+Cell vs. SCI+Medium, p=0.03). Surrounding the cavity were reactive microglia, identified by IBA1+ and astrocytes, revealed by staining for GFAP, displaying enlarged cell bodies and processes comparing to cells away from the injury site. Thus, our spinal cord injury model showed the pathological changes similar to those previously reported.

The transplanted human cells, identified by their positive staining for hNu and mCherry, were present in all the grafted animals (FIG. 4C). Stereological analysis indicated a total of 0.89±0.04×10⁶ hNu+ cells at 6 weeks and 1.06±0.08×10⁶ at 12 weeks post-transplantation (FIG. 4D). Examination of cell proliferation using immunostaining for Ki67 showed that 1.98%±0.04% of the hNu+ grafted cells were positive at 6 weeks and 0.72%±0.09% were positive by 3 months post-transplantation (FIG. 4E). The proportion of Ki67+ cells in the spinal cord of sham animals was 0.65±0.03% (FIG. 4E). These results indicated that the grafted human dl4 cells survived and their proliferation rate declined to the level similar to the endogenous cells.

Analysis of the (hNu+) cells indicated that the grafted cells were distributed in and around the injured area, mainly in the grey matter (FIG. 4C). At six weeks, 47.71% of the grafted cells were positive for NeuN. The remainders were positive for DCX and no cells for SOX9 (for astrocytes) and myelin basic protein (MBP, for oligodendrocytes). By 12 weeks after transplantation, 98%±1.7% of the grafted cells were positive for NeuN while no human cells were DCX positive (FIG. 4F and FIG. 4G), and 3.47%±0.93% were positive for SOX9 and there were no MBP positive cells.

Co-immunostaining for neuronal subtype markers indicated that 92.8%±4.73% of the human cells (hNu+, Stem121+) were positive for GABA (FIG. 4H, FIG. 4I). Further analysis of the grafted human cells indicated that 95.5%±2.01% human GABA were PTF1A positive (FIG. 4J) and 51%±4.02% were positive for LHX1/5, a dl4 domain neuron marker. These results indicated that the transplanted dl4 progenitors primarily differentiated to GABA neurons.

### Grafted Human Neurons Synapse with Host Spinal dl4s

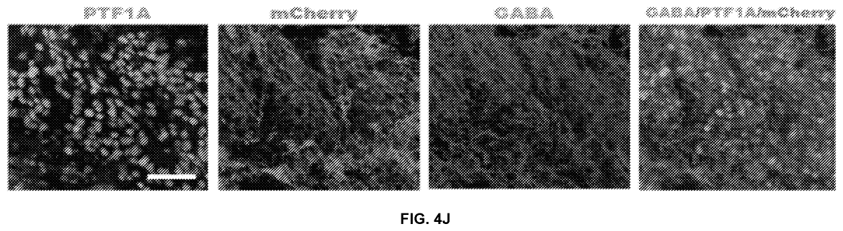

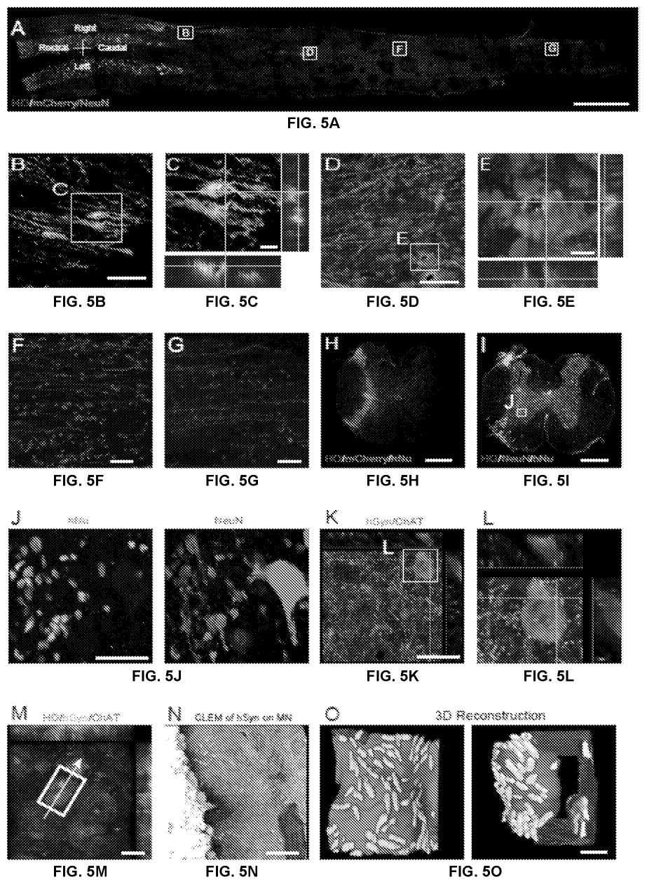

The somatosensory GABA neurons synapse with spinal dl4s to regulate spinal dl4 activity and hence muscle tone. Immunostaining for mCherry on horizontal sections (FIG. 5A-FIG. 5G) and cross sections (FIG. 5H-FIG. 5L) showed that grafted human neurons and their axons were present not only in the transplant site (T10/11) but also the lumbar segments. The mCherry+/hNu+ grafted cells were mostly in and near the grey matter at the transplant site (FIG. 4C, FIG. 5H-FIG. 5L). This was also seen in longitudinal sections that cut through the grey matter. Almost all the mCherry+ or hNu+ cells were positive for NeuN (FIG. 5B, FIG. 5C, FIG. 5H, FIG. 5J), and some were near the host spinal dl4s, indicated by the large size and NeuN+ but hNu− (FIG. 5J). The mCherry+ human axons were better viewed on the longitudinal sections, traveling to the lumbar cord mostly in the white matter (FIG. 5A-FIG. 5G) and fewer in grey matter. These results indicated the grafted cells migrated around the injury site and matured, and their axons travelled down to the lumbar cord mainly along the white matter.

Immunostaining for human-specific synaptophysin (hSyn) and ChAT showed numerous hSyn punctae on the surface of ChAT+ spinal dl4s, confirmed by z-stacks (0.5 μm step size) under the super resolution confocal analysis (FIG. 5K, FIG. 5L). This was also observed in horizontal sections where mCherry+ and hSyn+ punctae on a large NeuN+ but mCherry− host neuron. To further validate the synaptic connections between grafted human cells and endogenous rat spinal dl4s, we performed correlative light and electron microscopy on the focused area (FIG. 5M). As shown, multiple synaptic ends were on the surface of the spinal dl4 (FIG. 5M, FIG. 5N). Reconstruction of serial EM sections revealed numerous synaptic bouttons on the spinal dl4 surface (FIG. 5O). Thus, grafted human neurons formed synapses with host spinal dl4s.

### Transplantation with Spinal Inhibitory Neurons Alleviates Spasticity

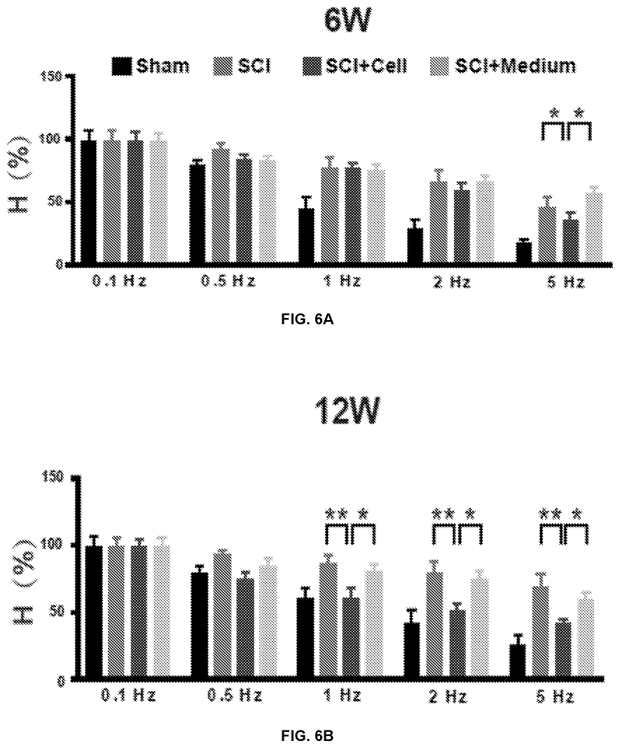

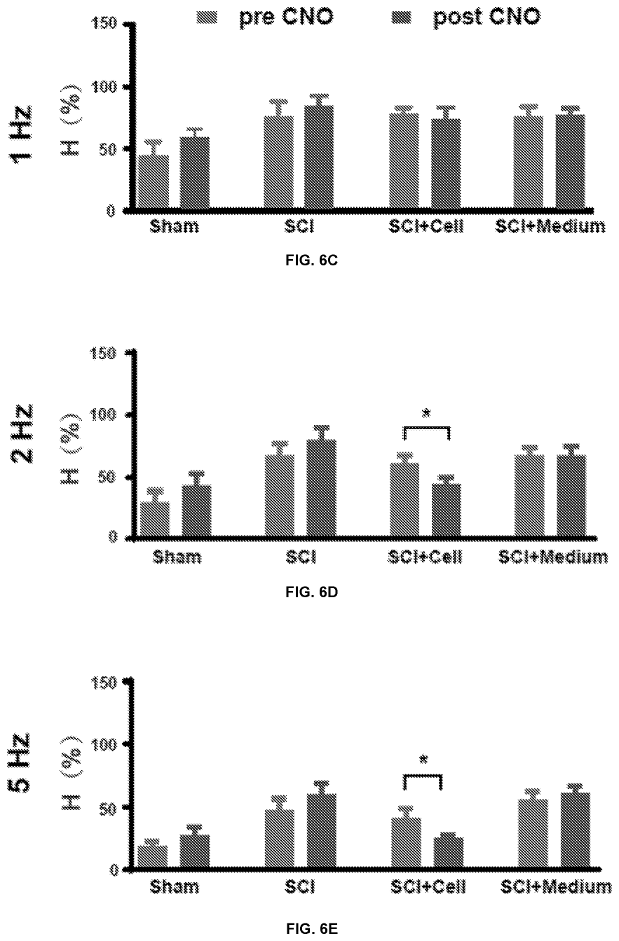

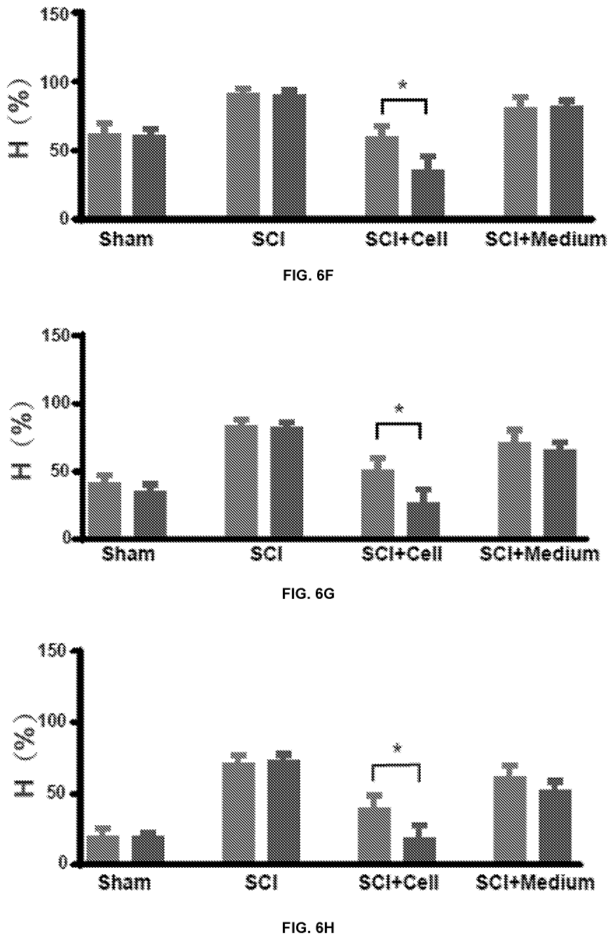

Spasticity was assessed by measuring the amplitude of the Hoffman reflex (H-reflex) which evaluates the function of spinal inhibitory systems (Jolivalt et al. Pain 2008, 140, 48-57; Kakinohana et al. Neuroscience 2006, 141, 1569-1583. The amplitude of H-reflex declines when the stimulus frequency reaches or exceeds 1 Hz, and that response is impaired in patients or animals with SCI. As predicted, the mean relative amplitude of H-reflex (H %) was significantly smaller in sham-operated rats when the stimulation frequency increased from 0.1 Hz to 5 Hz (100%±8.76% , 80.82%±8.24%, 46.06%±9.66%, 29.61% ±9.00%, and 10.45%±4.27% for 0.1, 0.5, 1, 2, and 5 Hz, respectively). All the other groups with SCI did not show obvious decline in H-reflex at six weeks post-transplant (FIG. 6A). However, when the stimulation frequency reached 5 Hz, the cell-transplanted animals showed reduction in the H-reflex (36.35%±7.46%) whereas the medium-injection group did not (58.05%±5.81%) (FIG. 6A). This suggested that transplantation with GABA neurons mitigated spasticity at 6 week post transplantation only when the stimulation was strong.

At 12 weeks post-transplantation, the cell-transplant group showed a similar decline in the H-reflex as the sham-operated animals, whereas the SCI group and medium-injection group did not show obvious change with an increasing stimulation frequency from 0.1 to 5 Hz (FIG. 6B). These results suggested that transplantation of GABA interneurons mitigated spasticity at a later stage.

Alleviation of spasticity by neural cell transplantation may be attributed to multiple factors, from trophic support to modulation of inflammation and repair of neural circuits, as suggested by measuring pain and locomotion. Taking advantage of the DREADD-expressing neural cells we transplanted, we asked if the therapeutic effects depend on the activity of the grafted cells. As shown above, spasticity, measured by the mean relative amplitude of H-reflex, showed no obvious change at 6 weeks post-transplantation (FIG. 6A). Interestingly, following CNO injection (3 mg/kg), the H % decreased to 47%±4.12% at 2 Hz and 28.21%±5.63% at 5 Hz of stimulation though not at 1 Hz (FIG. 6C, FIG. 6D, and FIG. 6E). At 12 weeks post-transplantation, CNO induced a decrease of H % from 59.99%±8.28% to 26.05%±5.64% even at 1 Hz (p=0.0475, FIG. 6F). Similar changes were observed at 2 Hz (50.85%±8.86% to 17.57%±4.98%, p=0.0286, FIG. 6G) and 5 Hz (40.31%±8.13% to 9.09%±1.52%, p=0.0346, FIG. 6H). As controls, animals with sham operation, SCI, and SCI injected with medium did not show any changes in response to CNO treatment (FIG. 6C-FIG. 6H). These results suggested that the transplanted GABA neurons, by regulating the spinal dl4 activity, alleviated spasticity.

### Transplantation with Spinal Inhibitory Neurons Improves Locomotion

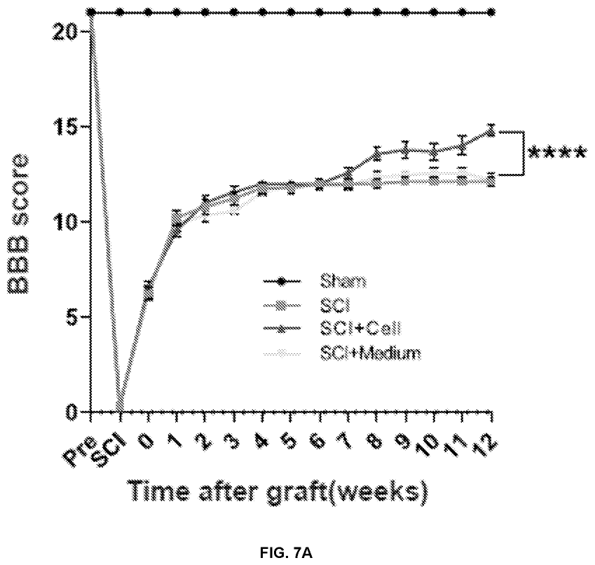

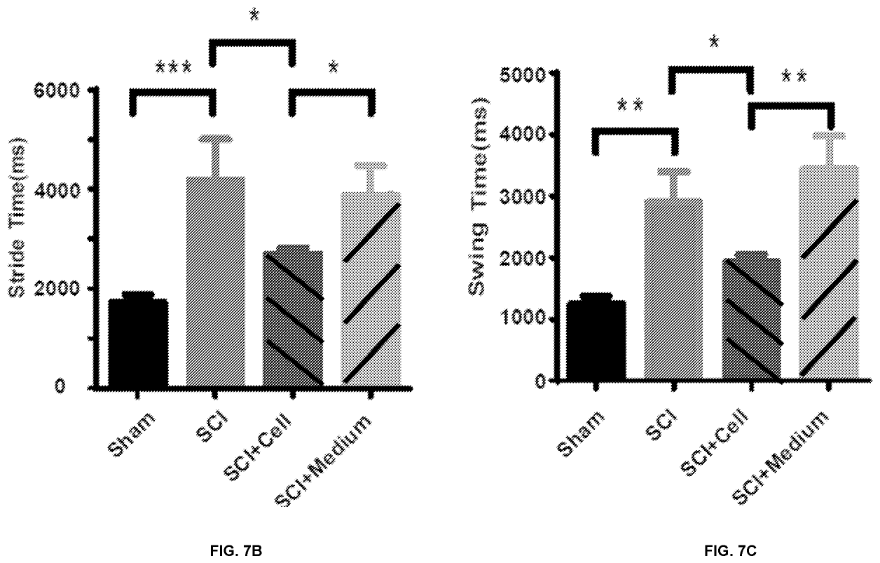

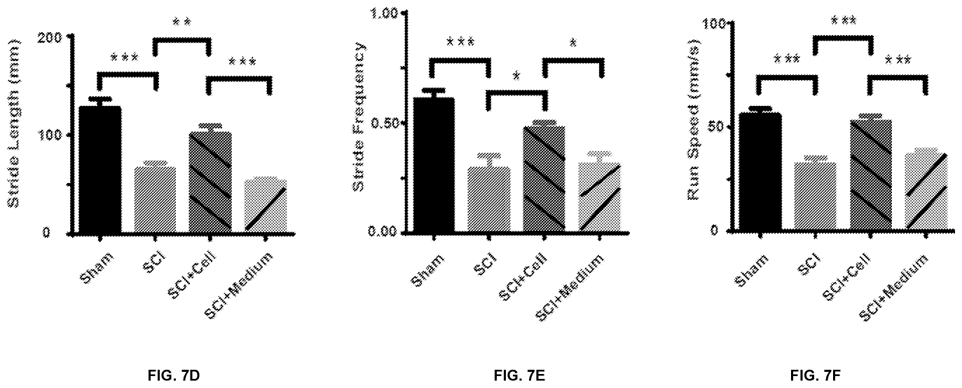

Spasticity affects gait and mobility. We measured locomotion weekly by an open field BBB score (Basso et al. Exp. Neurol. 1996, 139, 244-256) and unbiased Treadscan analysis at 12 weeks (Chen et al. J. Clin. Invest. 2015, 125, 1033-1042; Ma et al. Cell Stem Cell 2012, 10, 455-464; Qian et al. Stem Cell Reports 2017, 8, 843-855). The BBB scores dropped upon injury and were bounced back and stabilized from 2-3 weeks post-injury (FIG. 7A), suggesting a stable SCI. Animals with medium injection did not show an obvious difference from the those with SCI. The animals receiving cell transplantation showed improved BBB scores from 8 to 12 weeks post transplantation (SCI+Cell vs SCI+ Medium, P<0.0001), suggesting improved locomotion.

Since there are limited assays available to assess spasticity, we performed unbiased Treadscan to measure locomotion with an intention to identify additional parameters for predicting spasticity. Many parameters, including stride time, swing time, stride length, stride frequency of the limbs, and running speed may be used as parameters for measuring gaits and mobility. Stride length is determined as the average anterior-posterior distance between the heel markers at two consecutive heel strikes on the same side. Stride frequency (strides/s) is defined as the inverse of the interval between heel strikes of the same foot. The running speed is the average of all the instantaneous running speeds of strides.

Following SCI, the stride time and swing time of the hindlimbs increased (FIG. 7B, FIG. 7C), whereas stride length and stride frequency (FIG. 7D, FIG. 7E) decreased compared with the sham-operated group (FIG. 7B-FIG. 7E). Consequently, the run speed was slower (FIG. 7F). Animals with cell transplantation, but not with medium injection, reversed these parameters and their run speed was faster than those receiving medium injection (FIG. 7B-FIG. 7F), suggesting that cell transplantation alleviated spasticity and improved locomotion.

### Transplantation with Spinal Inhibitory Neurons Alleviates Neuropathic Pain

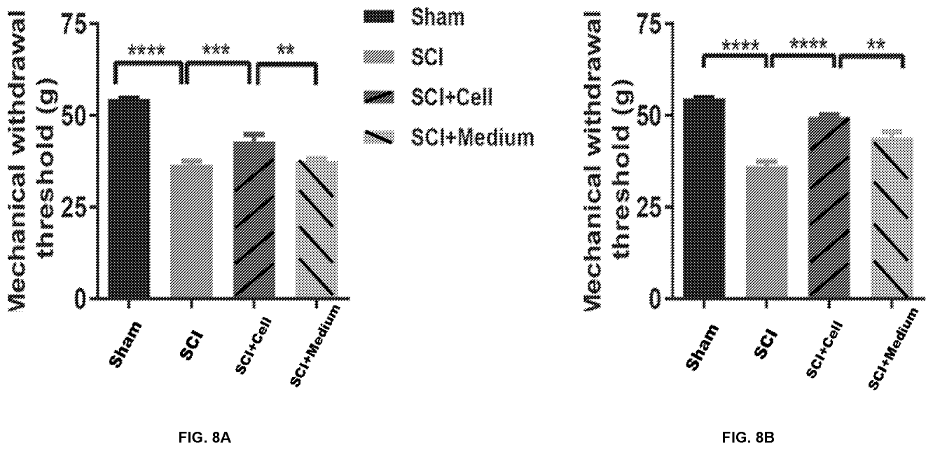

Neuropathic pain was assessed by measuring mechanical withdrawal of hindlimbs upon stimulation using an electronic von Frey apparatus (Electronic Von Frey Anesthesiometer 2450; IITC Life Sciences Inc). There was a significant decline in mechanical withdrawal threshold in the animals with SCI as compared to those with sham-operated (only laminectomy without SCI, 36.39 ±1.07 g vs. 54.50 ±0.20 g, p<0.0001), and there was an increased mechanical withdrawal threshold in stem cell transplantation group as compared to SCI+ medium group (42.87±1.90 g in SCI+Cell vs 37.47±0.75 g SCI+Medium, p=0.0084) at 6 weeks (FIG. 8A). A similar trend was observed at 12 weeks post-transplant (FIG. 8B). Thus, transplantation with the spinal GABA interneurons alleviated neuropathic pain in the SCI model.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A method of generating a population of spinal dl4 progenitor cells from human stem cells, the method comprising: (a) culturing the human stem cells in a culture medium comprising a Wnt signaling pathway agonist, a BMP signaling pathway inhibitor, and a TGFβ signaling pathway inhibitor, to generate a population of cells comprising spinal neuroepithelia progenitors, wherein at least 95% of the population of cells generated are Sox1+/Hoxa3+ spinal neuroepithelia progenitors; and (b) culturing the spinal neuroepithelia progenitors of step (a) in a medium additionally comprising a retinoic acid (RA) signaling agonist and a SHH signaling inhibitor, to generate a population of cells comprising spinal dl4 progenitor cells, wherein at least 90% of the population of cells generated are PTF1A+/PAX7+/ASCL1+ spinal dl4 progenitor cells.

Clause 2. The method of clause 1, wherein the human stem cells are human pluripotent stem cells.

Clause 3. The method of clause 2, wherein the human pluripotent stem cells are human embryonic stem cells or human induced pluripotent stem cells.

Clause 4 The method of any one of clauses 1-3, wherein the human stem cells are obtained from a human embryo.

Clause 5. The method of any one of clauses 1-4, wherein the Wnt signaling pathway agonist comprises a GSK3 inhibitor selected from the group consisting of CHIR99021 and 6-bromo-iridium-3′-oxime.

Clause 6. The method of any one of clauses 1-5, wherein the BMP signaling pathway inhibitor is selected from the group consisting of DMH-1, Dorsomorphin, and LDN-193189.

Clause 7. The method of any one of clauses 1-6, wherein the TGFβ signaling pathway inhibitor is selected from the group consisting of SB431542, SB505124, and A83-01.

Clause 8. The method of any one of clauses 1-7, wherein the RA signaling agonist is selected from the group consisting of retinoic acid (RA) and EC23.

Clause 9. The method of any one of clauses 1-8, wherein the SHH signaling inhibitor is selected from the group consisting of cyclopamine and SANT-1.

Clause 10. The method of any one of clauses 1-9, wherein the culture medium of step (a) comprises 2 μM SB431542, 2 μM DMH1, and 3 μM CHIR99021.

Clause 11. The method of any one of clauses 1-10, wherein the human stem cells are cultured in the culture medium in step (a) for 5 to 10 days.

Clause 12. The method of clause 11, wherein the human stem cells are cultured in the culture medium in step (a) for 7 days.

Clause 13. The method of any one of clauses 1-12, wherein the culture medium of step (b) comprises DMH-1, SB431542, CHIR99021, all-trans retinoic acid, and cyclopamine.

Clause 14. The method of any one of clauses 1-13, wherein the culture medium of step (b) comprises about 1 μM-10 μM DMH-1, about 1 μM-10 μM SB431542, about 1 μM-10 μM CHIR99021, about 0.01 μM-2 μM RA, and about 0.1 μM-2 μM cyclopamine.

Clause 15. The method of any one of clauses 1-14, wherein the culture medium of step (b) comprises 2 μM SB431542, 2 μM DMH1, 3 μM CHIR99021, 0.1 μM all-trans retinoic acid, and 0.5 μM cyclopamine.

Clause 16. The method of any one of clauses 1-15, wherein the spinal neuroepithelia progenitors are cultured in the culture medium in step (b) for 5 to 10 days.

Clause 17. The method of clause 16, wherein the spinal neuroepithelia progenitors are cultured in the culture medium in step (b) for 7 days.

Clause 18. The method of any one of clauses 1-17, wherein the spinal dl4 progenitor cells of step (b) express at least one gene selected from PTF1A, PAX7, and ASCL1, or a combination thereof.

Clause 19. The method of any one of clauses 1-18, wherein at least a portion of the spinal dl4 progenitor cells of step (b) further differentiate into spinal GABA interneurons.

Clause 20. The method of clause 19, wherein the spinal GABA interneurons express at least one gene selected from PAX2 and LHX1/5 or a combination thereof.

Clause 21. A method for treating a neurological disorder in a subject, the method comprising administering to the subject an effective amount of a population of spinal dl4 progenitor cells generated by the method of any one of clauses 1-20.

Clause 22. The method of clause 21, wherein the neurological disorder comprises a spinal cord injury.

1. A method of generating a population of spinal dl4 progenitor cells from human stem cells, the method comprising:
(a) culturing the human stem cells in a culture medium comprising a Wnt signaling pathway agonist, a BMP signaling pathway inhibitor, and a TGFβ signaling pathway inhibitor, to generate a population of cells comprising spinal neuroepithelia progenitors, wherein at least 95% of the population of cells generated are Sox1+/Hoxa3+ spinal neuroepithelia progenitors; and
(b) culturing the spinal neuroepithelia progenitors of step (a) in a medium additionally comprising a retinoic acid (RA) signaling agonist and a SHH signaling inhibitor, to generate a population of cells comprising spinal dl4 progenitor cells, wherein at least 90% of the population of cells generated are PTF1A+/PAX7+/ASCL1+ spinal dl4 progenitor cells.

2. The method of claim 1, wherein the human stem cells are human pluripotent stem cells.

3. The method of claim 2, wherein the human pluripotent stem cells are human embryonic stem cells or human induced pluripotent stem cells.

4. The method of claim 1, wherein the human stem cells are obtained from a human embryo.

5. The method of claim 1, wherein the Wnt signaling pathway agonist comprises a GSK3 inhibitor selected from the group consisting of CHIR99021 and 6-bromo-iridium-3′-oxime.

6. The method of claim 1, wherein the BMP signaling pathway inhibitor is selected from the group consisting of DMH-1, Dorsomorphin, and LDN-193189.

7. The method of claim 1, wherein the TGFβ signaling pathway inhibitor is selected from the group consisting of SB431542, SB505124, and A83-01.

8. The method of claim 1, wherein the RA signaling agonist is selected from the group consisting of retinoic acid (RA) and EC23.

9. The method of claim 1, wherein the SHH signaling inhibitor is selected from the group consisting of cyclopamine and SANT-1.

10. The method of claim 1, wherein the culture medium of step (a) comprises 2 μM SB431542, 2 μM DMH1, and 3 μM CHIR99021.

11. The method of claim 1, wherein the human stem cells are cultured in the culture medium in step (a) for 5 to 10 days.

12. The method of claim 11, wherein the human stem cells are cultured in the culture medium in step (a) for 7 days.

13. The method of claim 1, wherein the culture medium of step (b) comprises DMH-1, SB431542, CHIR99021, all-trans retinoic acid, and cyclopamine.

14. The method of claim 1, wherein the culture medium of step (b) comprises about 1 μM-10 μM DMH-1, about 1 μM-10 μM SB431542, about 1 μM-10 μM CHIR99021, about 0.01 μM-2 μM RA, and about 0.1 μM-2 μM cyclopamine.

15. The method of claim 1, wherein the culture medium of step (b) comprises 2 μM SB431542, 2 μM DMH1, 3 μM CHIR99021, 0.1 μM all-trans retinoic acid, and 0.5 μM cyclopamine.

16. The method of claim 1, wherein the spinal neuroepithelia progenitors are cultured in the culture medium in step (b) for 5 to 10 days.

17. The method of claim 16, wherein the spinal neuroepithelia progenitors are cultured in the culture medium in step (b) for 7 days.

18. The method of claim 1, wherein the spinal dl4 progenitor cells of step (b) express at least one gene selected from PTF1A, PAX7, and ASCL1, or a combination thereof.

19. The method of claim 1, wherein at least a portion of the spinal dl4 progenitor cells of step (b) further differentiate into spinal GABA interneurons.

20. The method of claim 19, wherein the spinal GABA interneurons express at least one gene selected from PAX2 and LHX1/5 or a combination thereof.

21. A method for treating a neurological disorder in a subject, the method comprising administering to the subject an effective amount of a population of spinal dl4 progenitor cells generated by the method of claim 1.

22. The method of claim 21, wherein the neurological disorder comprises a spinal cord injury.
